(19) United States
(12) Patent Application Publication
(10) Pub. No.: US 2013/0325065 A1
(43) Pub. Date: Dec. 5, 2013

(54) SPINOUS PROCESS CERCLAGE FOR BONE GRAFT CONTAINMENT

(71) Applicant: Simpirica Spine, Inc., San Carlos, CA (US)

(72) Inventors: Hugues Malandain, Mountain View, CA (US); Jeffrey Schwardt, Palo Alto, CA (US); Austin F. Noll, III, Palo Alto, CA (US); Ian Bennett, San Francisco, CA (US); Louis Fielding, San Carlos, CA (US)

(73) Assignee: Simpirica Spine, Inc., San Carlos, CA (US)

(21) Appl. No.: 13/963,770

(22) Filed: Aug. 9, 2013

Related U.S. Application Data
(63) Continuation of application No. PCT/US2012/025967, filed on Feb. 21, 2012.
(60) Provisional application No. 61/445,410, filed on Feb. 22, 2011.

(51) Int. Cl. A61B 17/70

(52) U.S. Cl. CPC A61B 17/7053; A61B 17/70
USPC 606/248; 606/263; 606/279

(57) ABSTRACT

A system for fusing a spine comprises a flexion limiting tether having a superior portion, and an inferior portion. The superior portion of the device is coupled to a superior portion of the spine, and the inferior portion of the device is coupled to an inferior portion of the spine thereby constraining flexion of the spine. The system also includes bone graft for fusing the superior and inferior portions of the spine together. The bone graft is disposed between the superior and inferior portions of the spine, and the tether has a width suitable for holding the bone graft in a mass disposed between the superior and inferior portions of the spine. The tether also has a porosity suitable to allow body fluids to pass therethrough so that the graft material forms a solid mass.

### CROSS-REFERENCE

This application is a continuation of International PCT Patent Application No. PCT/US2012/025967 (Attorney Docket No. 41564-718.601), filed Feb. 21, 2012 which is a non-provisional of and claims the benefit of U.S. Provisional Application No. 61/445,410 (Attorney Docket No. 41564-718.101), filed Feb. 22, 2011; the entire contents of which is incorporated herein by reference.

This application is related to the following U.S. patents and U.S. patent applications: U.S. Pat. No. 8,029,541; U.S. patent application Ser. Nos. 12/426,119, 12/721,198, 12/721,238, and 13/206,339, the entire contents of which are incorporated herein by reference.

### BACKGROUND OF THE INVENTION

1. Field of the Invention

The present invention generally relates to medical methods and apparatus. More particularly, the present invention relates to orthopedic internal fixation such as methods, devices, and accessories for restricting spinal flexion in patients having back pain or instability, or other orthopedic applications where a tether may be employed and other uses that the tether structure may advantageously provide.

A major source of chronic low back pain is discogenic pain, also known as internal disc disruption. Patients suffering from discogenic pain tend to be young, otherwise healthy individuals who present with pain localized to the back. Discogenic pain usually occurs at the discs located at the L4-L5 or L5-S1 junctions of the spine. Pain tends to be exacerbated when patients put their lumbar spines into flexion (i.e. by sitting or bending forward) and relieved when they put their lumbar spines into extension (i.e. by standing or arching backwards). Flexion and extension are known to change the mechanical loading pattern of a lumbar segment. When the segment is in extension, the axial loads borne by the segment are shared by the disc and facet joints (approximately 30% of the load is borne by the facet joints). In flexion, the segmental load is borne almost entirely by the disc. Furthermore, the nucleus shifts posteriorly, changing the loads on the posterior portion of the annulus (which is innervated), likely causing its fibers to be subject to tension and shear forces. Segmental flexion, then, increases both the loads borne by the disc and causes them to be borne in a more painful way. Discogenic pain can be quite disabling, and for some patients, can dramatically affect their ability to work and otherwise enjoy their lives.

Pain experienced by patients with discogenic low back pain can be thought of as flexion instability, and is related to flexion instability manifested in other conditions. The most prevalent of these is spondylolisthesis, a spinal condition in which abnormal segmental translation is exacerbated by segmental flexion. Flexion instability may be surgically-induced during common procedures such as neural decompression for spinal stenosis. This iatrogenic flexion instability may lead to back pain or recurrence of neurological symptoms. The methods and devices described should as such also be useful for these other spinal disorders or treatments associated with segmental flexion, for which the prevention or control of spinal segmental flexion is desired. Another application for which the methods and devices described herein may be used is in conjunction with a spinal fusion, in order to restrict motion, promote graft fusion and healing, and relieve pain post-operatively. Alternatively, the methods and devices described should also be useful in conjunction with other treatments of the anterior column of the spine, including kyphoplasty, total disc replacement, nucleus augmentation and annular repair. General orthopedic or surgical applications are envisioned where screw, rod, or plate fixation; bone fusion cages; or a tether, cable or tape may be employed.

Patients with discogenic pain accommodate their syndrome by avoiding positions such as sitting, which cause their painful segment to go into flexion, preferring positions such as standing, which maintain their painful segment in extension. One approach to reducing discogenic pain involves the use of a lumbar support pillow often seen in office chairs. Biomechanically, the attempted effect of the ubiquitous lumbar support pillow is also to maintain the painful lumbar segment in the less painful extension position. Postural and muscular compensation for spinal instability involves significant recruitment of the paraspinal musculature, and may exacerbate back pain.

Current treatment alternatives for patients diagnosed with chronic discogenic pain or flexion instability are quite limited. Many patients follow a conservative treatment path, such as physical therapy, massage, anti-inflammatory and analgesic medications, muscle relaxants, and epidural steroid injections, but typically continue to suffer with a significant degree of pain. Other patients elect to undergo spinal fusion surgery, which commonly requires discectomy (removal of the disk) together with fusion of adjacent vertebra. Fusion may or may not also include instrumentation of the affected spinal segment including, for example, pedicle screws and stabilization rods. Fusion is not usually recommended for discogenic pain because it is irreversible, costly, associated with high morbidity, and has questionable effectiveness. Despite its drawbacks, however, spinal fusion for discogenic pain remains common due to the lack of viable alternatives.

An alternative method, that is not commonly used in practice, but has been approved for use by the United States Food and Drug Administration (FDA), is the application of bone cerclage devices which can encircle the spinous processes or other vertebral elements and thereby create a restraint to motion. Physicians typically apply a tension or elongation to the devices that applies a constant and high force on the anatomy, thereby fixing the segment in one position and allowing effectively no motion. The lack of motion allowed after the application of such devices is thought useful to improve the likelihood of fusion performed concomitantly; if the fusion does not take, these devices will fail through breakage of the device or of the spinous process to which the device is attached. These devices are designed for static applications and are not designed to allow for dynamic elastic resistance to flexion across a range of motion. The purpose of bone cerclage devices and other techniques described above is to almost completely restrict measurable motion of the vertebral segment of interest. This loss of motion at a given segment gives rise to abdominal loading and motion at adjacent segments, which can lead eventually to adjacent segment morbidity.

Another solution involves the use of an elastic structure, such as tethers, coupled to the spinal segment. The elastic structures are typically secured to the spinal segment with pedicle screws, or sometimes tethers. The elastic structures can relieve pain by increasing passive resistance to flexion while often allowing substantially unrestricted spinal extension. This mimics the mechanical effect of postural accommodations that patients already use to provide relief.

Spinal implants using tether structures are currently commercially available. One such implant couples adjacent vertebrae via their pedicles. This implant includes spacers, tethers and pedicle screws. To install the implant, selected portions of the disc and vertebrae bone are removed. Implants are then placed to couple two adjacent pedicles on each side of the spine. The pedicle screws secure the implants in place. The tether is clamped to the pedicle screws with set-screws, and limits the extension/flexion movements of the vertebrae of interest. Because significant tissue is removed and because of screw placement into the pedicles, the implant and accompanying surgical methods are highly invasive and the implant is often irreversibly implanted. There is also an accompanying high chance of nerve root damage. Where the tip of the set-screw clamps the tethers, the tethers are abraded and may generate particulate debris.

Other implants employing tether structures couple adjacent vertebrae via their processes instead. These implants include a tether and a spacer. To install the implant, the supraspinous ligament is temporarily lifted and displaced. The interspinous ligament between the two adjacent vertebrae of interest is then permanently removed and the spacer is inserted in the interspinous interspace. The tether is then wrapped around the processes of the two adjacent vertebrae, through adjacent interspinous ligaments, and then mechanically secured in place by the spacer or also by a separate component fastened to the spacer. The supraspinous ligament is then restored back to its original position. Such implants and accompanying surgical methods are not without disadvantages. These implants may subject the spinous processes to frequent, high loads during everyday activities, sometimes causing the spinous processes to break or erode. Furthermore, the spacer may put a patient into segmental kyphosis, potentially leading to long-term clinical problems associated with lack of sagittal balance. The process of securing the tethers is often a very complicated maneuver for a surgeon to perform, making the surgery much more invasive. And, as previously mentioned, the removal of the interspinous ligament is permanent. As such, the application of the device is not reversible.

More recently, less invasive spinal implants have been introduced. Like the aforementioned implant, these spinal implants are placed over one or more pairs of spinous processes and provide an elastic restraint to the spreading apart of the spinous processes during flexion. However, spacers are not used and interspinous ligaments are not permanently removed. As such, these implants are less invasive and may be reversibly implanted. The implants typically include a tether and a securing mechanism for the tether. The tether may be made from a flexible polymeric textile such as woven polyester (PET) or polyethylene; multi-strand cable, or other flexible structure. The tether is wrapped around the processes of adjacent vertebrae and then secured by the securing mechanism. The securing mechanism may involve the indexing of the tether and the strap, e.g., the tether and the securing mechanism include discrete interfaces such as teeth, hooks, loops, etc. which interlock the two. Highly forceful clamping may also be used to press and interlock the tether with the securing mechanism. Many known implementations can clamp a tether with the tip of a set-screw, or the threaded portion of a fastener. However, the mechanical forces placed on the spinal implant are unevenly distributed towards the specific portions of the tether and the securing mechanism which interface with each other. These portions are therefore typically more susceptible to abrasion, wear, or other damage, thus reducing the reliability of these spinal implants as a whole. Other known methods use a screw or bolt to draw other components together to generate a clamping force. While these methods may avoid the potentially damaging loads, the mechanical complexity of the assembly is increased by introducing more subcomponents. Other methods use a buckle through which the tether is threaded in a tortuous path, creating sufficient friction to retain the tether. These buckles generally distribute the load over a length of the tether; although they may be cumbersome to use and adjust as the tether is required to be threaded around multiple surfaces and through multiple apertures. Many of the aforementioned methods involve the use of several components, which must often be assembled during the surgical procedure, often within the wound. This adds time, complexity and risk to the surgical procedure.

More recently, spinous process plate fusion devices have been introduced. These devices typically utilize spiked plates that clamp medially against the spinous processes to restrict flexion and extension motions of the spinal segment. Bone graft is often placed between the spinous processes to attain interspinous or interlaminar fusion. The plate type devices, however, may impose concentrated stresses on the spinous processes. Additionally, they do not compress the spinous processes together against the interpinous fusion graft. Such interspinous compression would promote fusion of the spinous processes and lamina.

For the aforementioned reasons, it would be desirable to provide improved methods and apparatus that allow flexion resisting tether devices to be used with other orthopedic treatments such as a spinal fusion procedure without requiring additional implants or instrumentation. Such improved methods and procedures will preferably allow the flexion resisting tether devices to be easily implanted, and to help facilitate spinal fusion by compressing the spinous processes together. In particular, such methods and apparatus should be minimally invasive and should enable the tether to be more easily, reversibly, repeatably, safely and reliably implanted and adjusted by a surgeon, in a surgery setting.

2. Description of the Background Art

Patents and published applications of interest include: U.S. Pat. Nos. 3,648,691; 4,643,178; 4,743,260; 4,966,600; 5,011,494; 5,092,866; 5,116,340; 5,180,393; 5,282,863; 5,395,374; 5,415,658; 5,415,661; 5,449,361; 5,456,722; 5,462,542; 5,496,318; 5,540,698; 5,562,737; 5,609,634; 5,628,756; 5,645,599; 5,725,582; 5,902,305; Re. 36,221; 5,928,232; 5,935,133; 5,964,769; 5,989,256; 6,053,921; 6,248,106; 7,163,558; Published U.S. Patent Application Nos. US 2002/0151978; US 2004/0024458; US 2004/0106995; US 2004/0116927; US 2004/0117017; US 2004/0127989; US 2004/0172132; US 2004/0243239; US 2005/0033435; US 2005/0049708; 2005/0192581; 2005/0216017; US 2006/0069447; US 2006/0136060; US 2006/0240533; US 2007/0213829; US 2007/0233096; Published PCT Application Nos. WO 01/28442 A1; WO 02/03882 A2; WO 02/051326 A1; WO 02/071960 A1; WO 03/045262 A1; WO2004/052246 A1; WO 2004/073532 A1; and Published Foreign Application Nos. EP0322334 A1; and FR 2 681 525 A1. The mechanical properties of flexible constraints applied to spinal segments are described in Papp et al. (1997) Spine 22:151-155; Dickman et al. (1997) Spine 22:596-604; and Gamer et al. (2002) Eur. Spine J. S186-S191; A1 Baz et al. (1995) Spine 20, No. 11, 1241-1244; Heller, (1997) Arch. Orthopedic and Trauma Surgery, 117, No. 1-2:96-99; Leahy et al. (2000) Proc. Inst. Mech. Eng. Part H: J. Eng. Med. 214, No. 5: 489-495; Minns et al., (1997) Spine 22 No. 16:1819-1825; Miyasaka et al. (2000) Spine 25, No. 6: 732-737; Shepherd et al. (2000) Spine 25, No. 3: 319-323; Shepherd (2001) Medical Eng. Phys. 23, No. 2: 135-141; and Voydeville et al (1992) Orthop Traumatol 2:259-264.

### BRIEF SUMMARY OF THE INVENTION

The present invention provides methods and apparatus for using flexion restricting tether devices in combination with a spinal fusion procedure preferably while minimizing or eliminating the need for spinal instrumentation such as pedicle screws and rods. Combining flexion restricting tether devices that also facilitate fusion may provide promising treatments for discogenic pain as well as other conditions, such as degenerative spondylolisthesis.

A first aspect of the present invention provides a system for fusing a spine. The system comprises a flexion limiting tether and a bone graft. The flexion limiting tether has a superior portion and an inferior portion. The superior portion of the tether is coupled to a superior portion of the spine and the inferior portion of the tether is coupled to an inferior portion of the spine, thereby constraining flexion of the spine. The bone graft is for fusing the superior and inferior portions of the spine together and is disposed between the superior and inferior portions of the spine. The tether has a width suitable for holding the bone graft in a mass disposed between the superior and inferior portions of the spine. The tether will typically have a porosity suitable to allow body fluids to pass therethrough so that material of the bone graft forms a solid mass. Typically, the superior portion of the spine comprises a superior spinous process and the inferior portion of the spine comprises an inferior spinous process, and the flexion limiting tether may be wide enough to cover a majority of the lateral surfaces of the superior and inferior spinous processes.

In many embodiments, the system further comprises a connector and the flexion limiting tether consists essentially of a single strap having a free end and a fixed end. The fixed end is fixedly coupled to the connector and the free end is adjustably coupled to the connector such that the tether can be tightened over the superior and inferior portions of the spine.

In some embodiments, the connector may comprise an inward facing surface having one or more spikes adapted to facilitate purchase of the connector onto the bone of the superior or inferior portion of the spine.

In some embodiments, the system may further comprise a plate adapted to be disposed on the other side of a spinal midline from the connector. The flexion limiting tether may be wrapped over the plate. The plate may comprise an inward facing surface having one or more spikes adapted to facilitate purchase of the plate onto the bone of the superior or inferior portion of the spine. The plate may comprise one or more cross-members adapted to traverse the spinal midline to couple to the connector. The one or more cross-members may be dorsal of the connector and the plate, may comprise a central cross-member for adjusting the distance between the connector and the plate, and/or may comprise a fixed cross-member and an adjustable cross-member, with the position of the adjustable cross-member relative to the fixed cross-member being adjustable so as to adjust the distance between the adjustable cross-member and the fixed cross-member and also optionally being adjustable to distract the superior and inferior portions of the spine.

In many embodiments, the superior portion of the tether comprises a first strap and the inferior portion of the tether comprises a second strap distinct from the first strap. Typically, the first strap comprises a fixed end and a free end, the second strap comprises a fixed and a free end, and the system further comprises a first connector and a second connector. The fixed end of the first strap will be fixedly coupled to the first connector and the free end of the first strap will be adjustably coupled to the first connector such that the first strap can be tightened over the superior portion of the spine. Likewise, the fixed end of the second strap will be fixedly coupled to the second connector and the free end of the second strap will be adjustably coupled to the second connector such that the second strap can be tightened over the inferior portion of the spine. The first connector and the second connector may be disposed on opposite sides of a spinal midline. The first connector may comprise an inward facing surface having one or more spikes adapted to facilitate purchase of the first connector to bone of the superior portion of the spine. The first connector may comprise an inward facing surface having one or more spikes adapted to facilitate purchase of the first connector to bone of the superior portion of the spine.

In some embodiments, the system may further comprise one or more cross-members adapted to traverse the spinal midline to couple the first connector to the second connector. The one or more cross-members may be dorsal of the first connector and the second connector. The one or more cross-members may comprise a central cross-member for adjusting the distance between the first connector and the second connector. The one or more cross-members may comprise a fixed cross-member and an adjustable cross-member, with the position of the adjustable cross-member relative to the fixed cross-member being adjustable so as to adjust the distance between the adjustable cross-member and the fixed cross-member and also optionally being adjustable to distract the superior and inferior portions of the spine.

In many embodiments, the system may further comprise a fusion cage for holding the bone graft in place. The fusion cage may comprise a cylindrical main body having a plurality of pores to allow body fluids to pass therethrough so that the material of the bone graft can form a solid mass.

In many embodiments, the system may further comprise one or more fasteners for piercing through the flexion limiting tether and into the bone graft to hold the flexion limiting tether in place relative to the bone graft.

Another aspect of the present invention provides a method for fusing a spine. A tether is provided. The tether is coupled to a superior portion of the spine and an inferior portion of the spine, and constrains flexion of the spine. A bone graft is provided. The bone graft is disposed the superior and inferior portions of the spine. The bone graft is constrained with the tether so that the bone graft is held in a mass. The tether is tightened to apply a compressive force to the bone graft via the superior and inferior portions of the spine. Typically, body fluids are allowed to pass through the tether into contact with the bone graft, thereby allowing the bone graft to form a solid mass. The superior portion of the spine will typically comprise a superior spinous process and the inferior portion of the spine will typically comprise an inferior spinous process. And, the method may further comprise a step of removing at least a portion of the interspinous ligament between the superior spinous process and the inferior spinous process prior to disposing the bone graft therebetween.

In many embodiments, a connector for the tether is provided. The connector is fixedly coupled to a fixed end of the tether and is adjustably coupled to an adjustable end of the tether. The position of the adjustable end of the tether relative to the connector is adjusted so as to loosen or tighten the tether over the bone graft and superior and inferior portions of the spine. One or more spikes on an inward facing surface of the connector may be provided. The one or more spikes facilitate purchase of the connector to bone of the superior or inferior portion of the spine.

In many embodiments, a fusion cage is further provided. The fusion cage holds the bone graft in place relative to the superior and inferior portions of the spine.

In many embodiments, the tether and bone graft is pierced with a fastener which is left in place through the tether and bone graft to hold the bone graft in place relative to the tether.

These and other embodiments are described in further detail in the following description related to the appended drawing figures.

### BRIEF DESCRIPTION OF THE DRAWINGS

FIG. 1 is a schematic diagram illustrating the lumbar region of the spine.

FIG. 1A a schematic illustration showing a portion of the lumbar region of the spine taken along a sagittal plane.

FIG. 2 illustrates a spinal implant of the type described in US 2005/0216017A1.

FIGS. 3A-3B illustrate additional tissue surrounding the spinous processes.

FIGS. 4A-4M show an exemplary method of surgically implanting a spinal device.

FIG. 5 illustrates an exemplary compliance element.

FIGS. 6A-6C illustrate the use of an exemplary fastening mechanism incorporated in the compliance element for removably locking a tether.

FIG. 7 is an exploded view of an exemplary fastening mechanism.

FIG. 8A illustrates a spinal implant comprising a single strap or tether structure and single connector or buckle.

FIGS. 8B-8N show an exemplary method of surgically implanting the spinal device of FIG. 8A.

FIGS. 9A-9B show an exemplary spinal implant comprising a single strap or tether structure held in place relative to a fusion cage or bone graft with a pair of spikes.

FIGS. 10A-10D show exemplary spinal implants comprising one or more straps and/or one or more cross-members.

FIGS. 11A-11D show an exemplary spinal fusion cage adapted to facilitate cerclage of the spinal processes with a strap or tether structure.

### DETAILED DESCRIPTION OF THE INVENTION

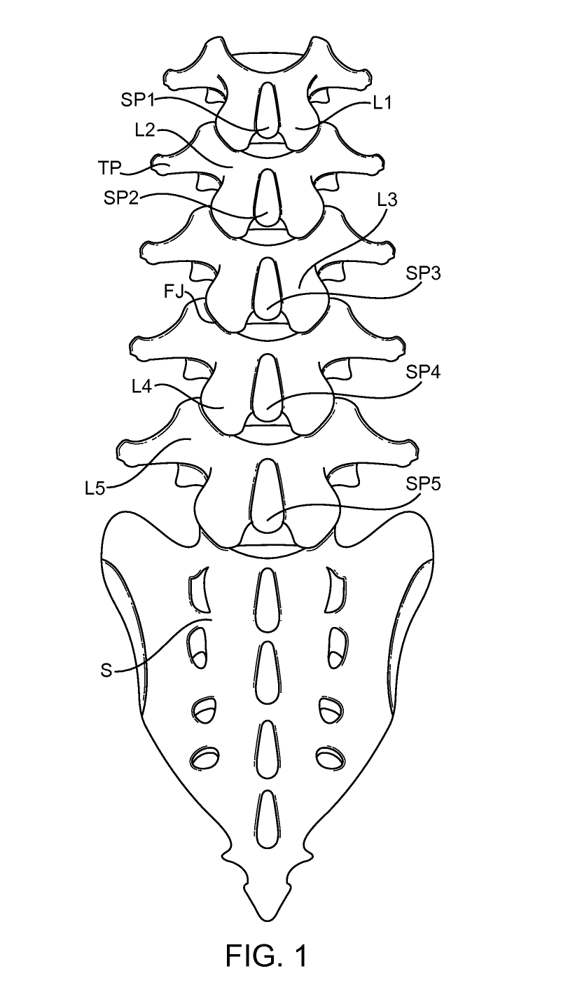

FIG. 1 is a schematic diagram illustrating the lumbar region of the spine including the spinous processes (SP), facet joints (FJ), lamina (L), transverse processes (TP), and sacrum (S).

FIG. 1A is a schematic illustration showing a portion of the lumbar region of the spine taken along a sagittal plane and is useful for defining the terms “neutral position,” “flexion,” and “extension” that are often used in this disclosure.

As used herein, “neutral position” refers to the position in which the patient's spine rests in a relaxed standing position. The “neutral position” will vary from patient to patient. Usually, such a neutral position will be characterized by a slight curvature or lordosis of the lumbar spine where the spine has a slight anterior convexity and slight posterior concavity. In some cases, the presence of the constraint of the present invention may modify the neutral position, e.g. the device may apply an initial force which defines a “new” neutral position having some extension of the untreated spine. As such, the use of the term “neutral position” is to be taken in context of the presence or absence of the device. As used herein, “neutral position of the spinal segment” refers to the position of a spinal segment when the spine is in the neutral position.

Furthermore, as used herein, “flexion” refers to the motion between adjacent vertebrae in a spinal segment as the patient bends forward. Referring to FIG. 1A, as a patient bends forward from the neutral position of the spine, i.e. to the right relative to a curved axis A, the distance between individual vertebrae L on the anterior side decreases so that the anterior portion of the intervertebral disks D are compressed. In contrast, the individual spinous processes SP on the posterior side move apart in the direction indicated by arrow B. Flexion thus refers to the relative movement between adjacent vertebrae as the patient bends forward from the neutral position illustrated in FIG. 1A.

Additionally, as used herein, “extension” refers to the motion of the individual vertebrae L as the patient bends backward and the spine extends from the neutral position illustrated in FIG. 1A. As the patient bends backward, the anterior ends of the individual vertebrae will move apart. The individual spinous processes SP on adjacent vertebrae will move closer together in a direction opposite to that indicated by arrow B.

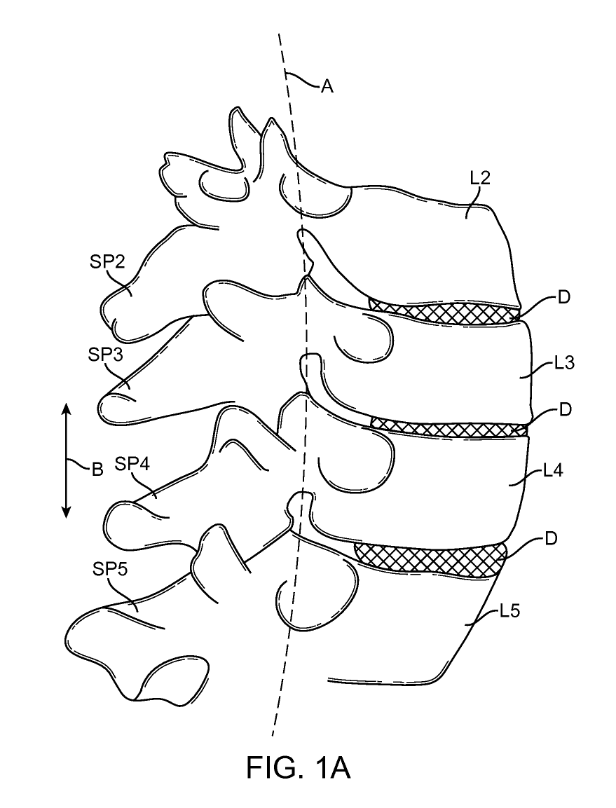

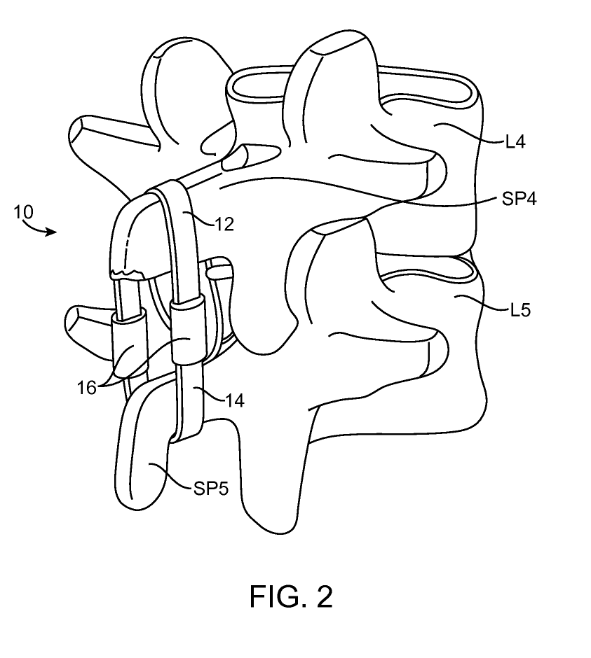

FIG. 2 shows a spinal implant of the type described in related U.S. Patent Publication No. 2005/0216017 A1 (now U.S. Pat. No. 7,458,981), the contents of which are herein incorporated by reference. As illustrated in FIG. 2, an implant 10 typically comprises an upper strap component 12 and a lower strap component 14 joined by a pair of compliance members 16. The upper strap 12 is shown disposed over the top of the spinous process SP4 of L4 while the lower strap 14 is shown extending over the bottom of the spinous process SP5 of L5. The compliance member 16 will typically include an element, such as a spring or rubber block, which is attached to the straps 12 and 14 in such a way that the straps may be “elastically” or “compliantly” pulled apart as the spinous processes SP4 and SP5 move apart during flexion. In this way, the implant provides an elastic tension on the spinous processes which provides a force that resists flexion. The force increases as the processes move further apart. Usually, the straps themselves will be essentially non-compliant so that the degree of elasticity or compliance may be controlled and provided solely by the compliance members 16.

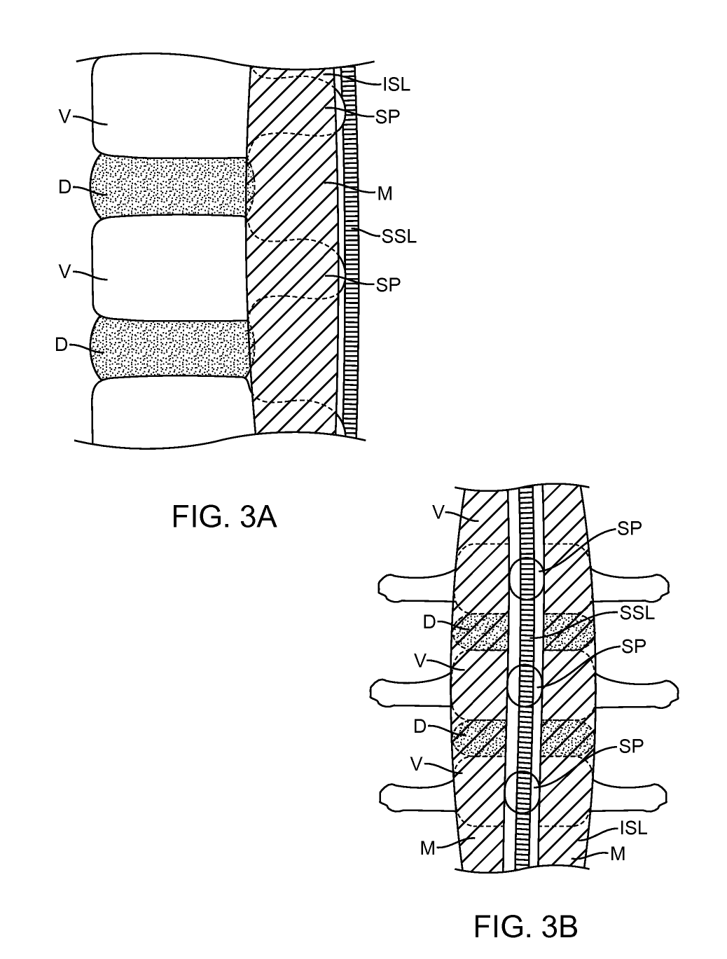

FIG. 3A is a side view of the lumbar region of the spine having discs D separating the vertebral bodies V. The supraspinous ligament SSL runs along the posterior portion of the spinous processes SP and the interspinous ligament ISL and multifidus tendon and muscle M run alongside of and attach to the spinous processes SP. FIG. 3B is a posterior view of FIG. 3A.

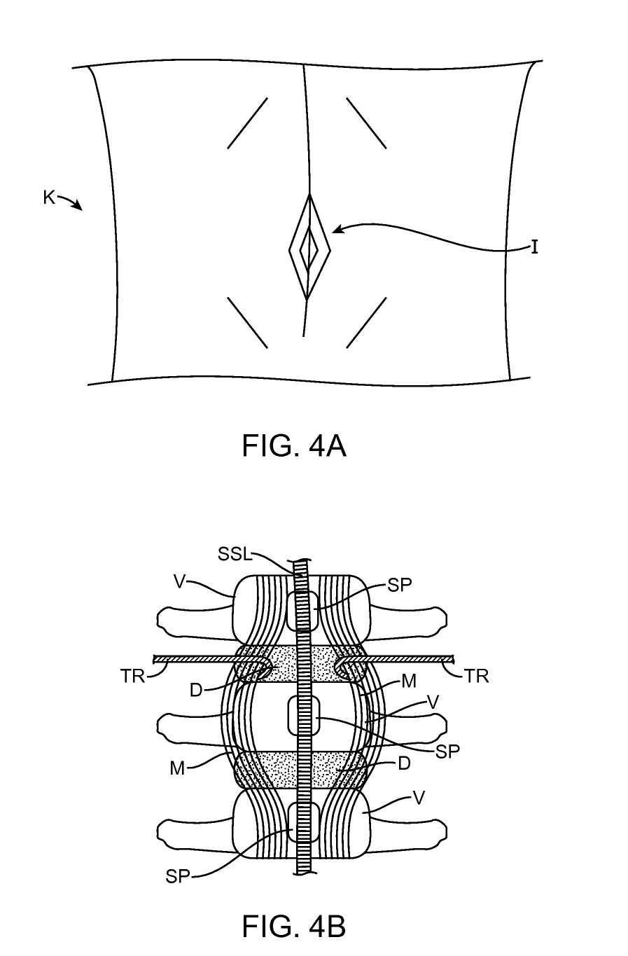

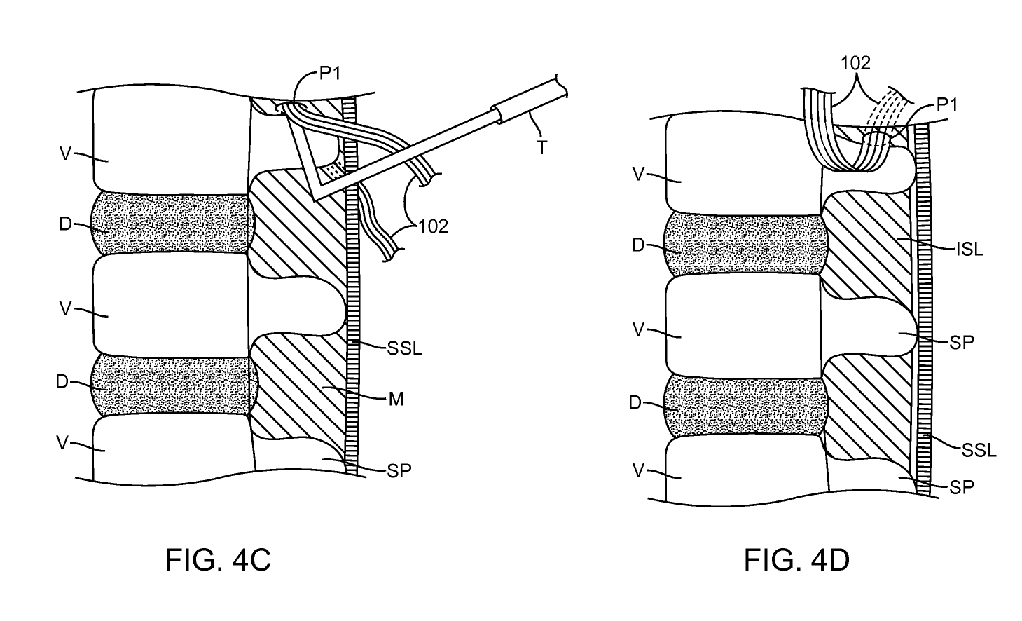

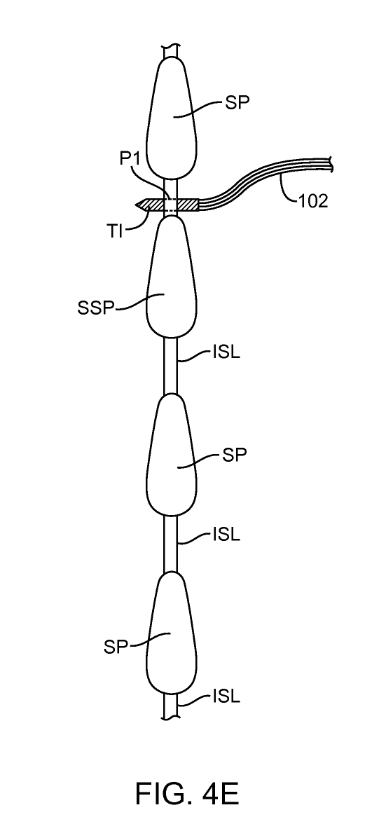

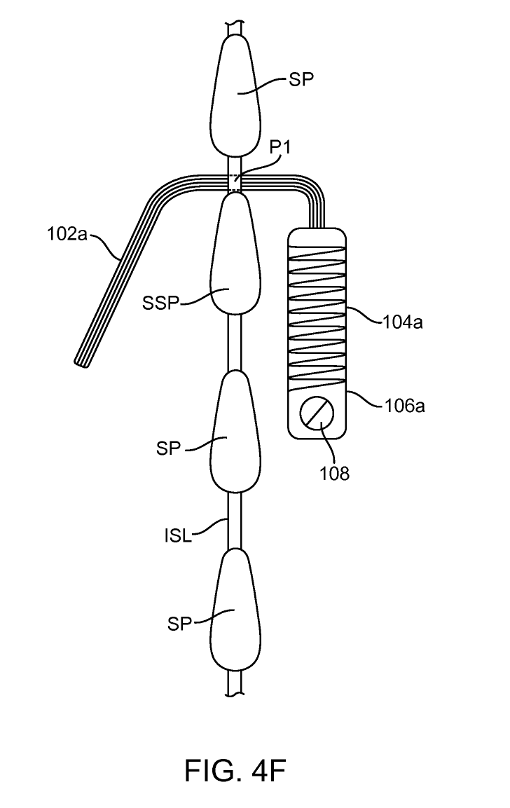

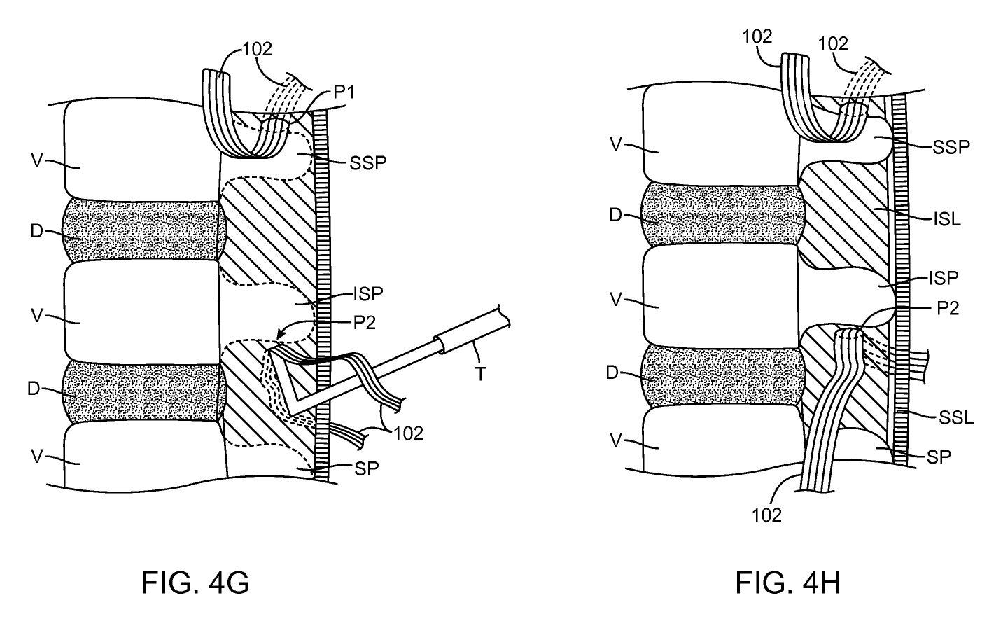

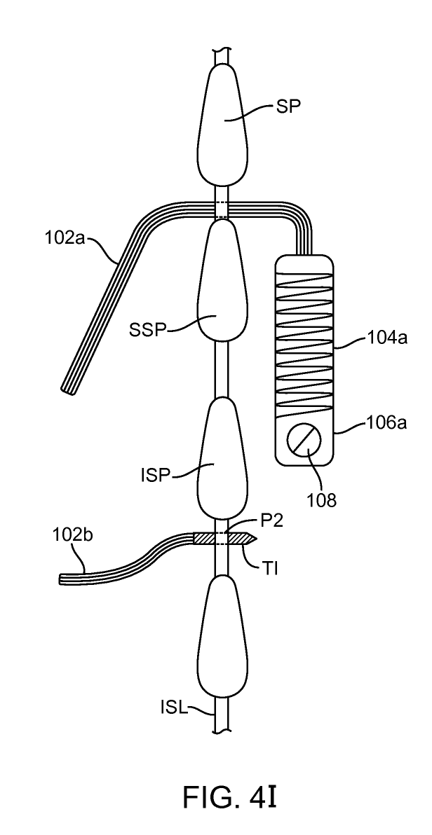

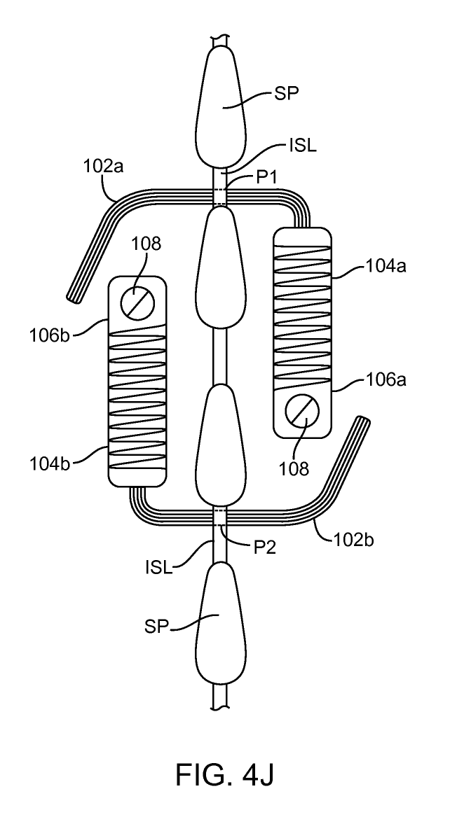

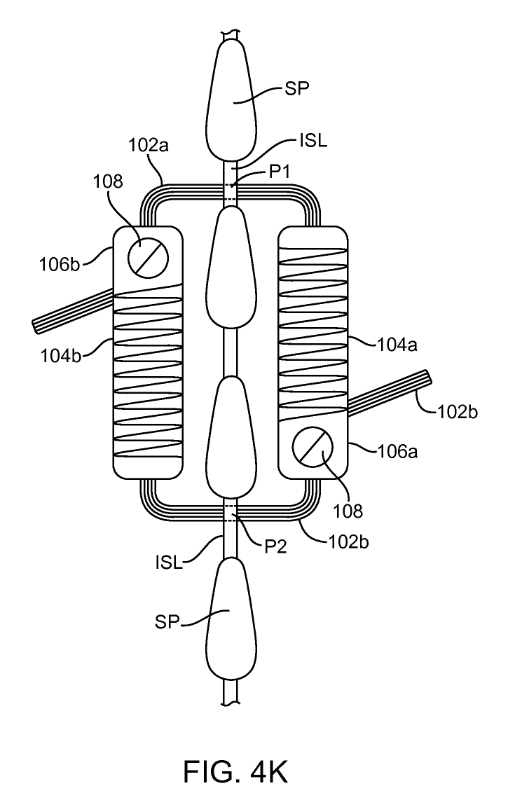

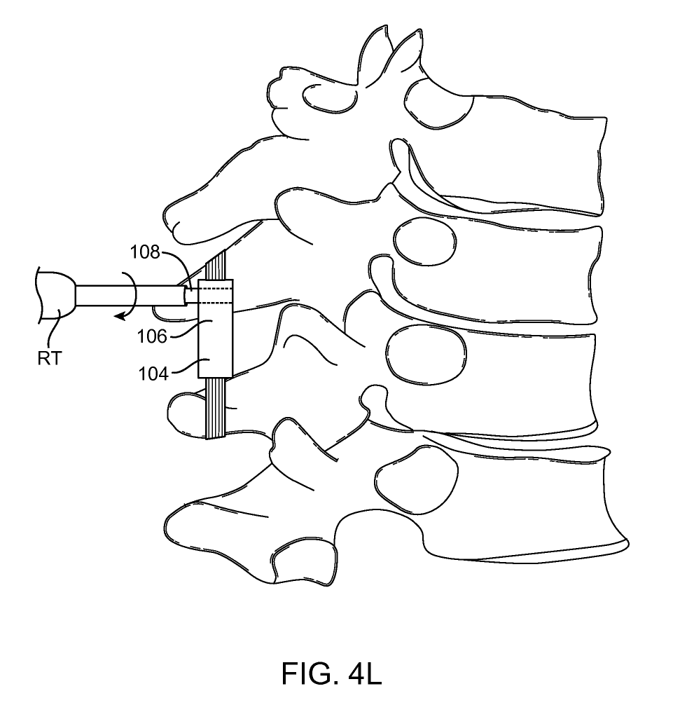

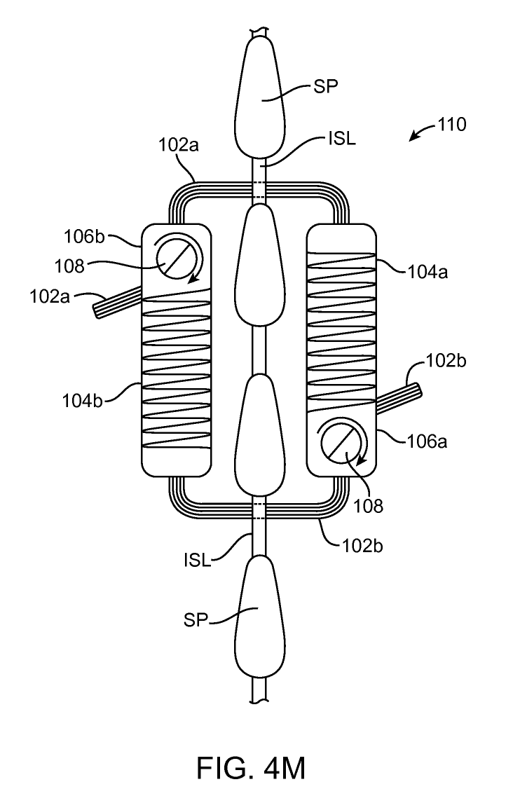

FIGS. 4A-4M illustrate an exemplary surgical method of implanting a spinous process constraint such as the embodiment of FIG. 2. One of the first steps to surgically implant a spinal implant is to make an incision to access the spinal area of interest. FIG. 4A shows the lumbar region of back K after an incision I has been made through the patient's skin. FIG. 4B illustrates the lumbar region of the spine after the incision I has been made through the patient's skin. Multifidus muscle and tendon M have been retracted with retraction tools TR to expose the spinous processes.

After the incision has been made, a piercing tool T having a sharp distal end may be used to access and pierce the interspinous ligament ISL while avoiding the supra spinous ligament SSL, creating an interspinous ligament perforation P1 superior of the first spinous process SSP of interest. This surgical approach is desirable since it keeps the supra spinous ligament intact and minimizes damage to the multifidus muscle and tendons and other collateral ligaments. As shown in FIG. 4C, from the right side of the spine, tool T accesses and pierces the interspinous ligament ISL adjacent of the first spinous process SSP of interest. The distal end of tool T is shown in dotted line. Alternatively, tool T may access and pierce the interspinous ligament ISL from the left side instead. The distal end of tool T is coupled with tether 102, parts of which are also shown in dotted line. In addition to accessing and piercing the interspinous ligament ISL, piercing tool T also advances or threads tether 102 through perforation P1. As shown in FIG. 4D, tool T is then removed, leaving tether 102 positioned through perforation P1. Multifidus tendon and muscle M is not shown in FIGS. 4C and 4D so that other elements are shown more clearly.

FIG. 4E is a posterior view of a section of the spine after the above steps have been performed. Often times, the distal tip TI of tool T is detachable. As shown in FIG. 4E, after tool T accesses and pierces the interspinous ligament ISL with distal tip TI, distal tip TI is detached from tool T and is left in place in perforation P1 (shown in dotted line) above the first spinous process SSP of interest. Tether 102 lags behind tip TI. In some cases, distal tip TI may fully pierce through interspinous ligament ISL. In these cases, distal tip TI has passed through the interspinous ligament ISL while a portion of tether 102 is left in place in perforation P1.

After tip TI or a portion of tether TH is left in place in perforation P1, another tool may couple with tip TI and pull tip TI such that it drags tether 102a and compliance element 104a to its appropriate position relative to the spine, as shown in FIG. 4F. Compliance element 104a is coupled to tether 102a and is used to provide a force resistive to flexion of spinous processes SP. Compliance element 104a includes a fastening mechanism or fastening element 106a and may further comprise a spring, a tensioning member, a compression member, or the like. Related compliance members are described in commonly owned U.S. patent application Ser. No. 12/106,103 (Attorney Docket No. 026398-000410US), the entire contents of which are incorporated herein by reference.

The steps of accessing the ISL, piercing the ISL, and threading tether 102 through a perforation are then repeated for the opposite, lateral side of the spine for an adjacent spinous process ISP, inferior of the first superior spinal process SSP of interest. As shown in FIGS. 4G and 4H, tool T accesses the interspinous ligament from the left side of the spinal midline and pierces the interspinous ligament ISL, creating a second perforation P2 located inferior of a second spinous process of interest, labeled as inferior spinous process ISP. As shown in FIG. 4G, the inferior spinous process ISP of interest is directly adjacent and inferior to the first superior spinous process SSP of interest. However, it is entirely possible to perform the described procedure starting with the inferior spinous process ISP first instead of the superior spinous process SSP, for example, perforation P2 may be created before perforation P1. It is also possible that there may be a gap of one or more spinous processes SP between the spinous processes of interest. Multifidus tendon and muscle M is not shown in FIGS. 4G and 4H for clarity of the other shown elements.

As shown in FIGS. 4H, 41 and 4J, like with the steps shown in conjunction with the first piercing, tether 102b is pierced through perforation P2 and left in place along with distal tip TI of tool T (best seen in FIG. 41). Another tool such as a pair of forceps, is then used to grasp distal tip TI to pull tether 102b and compliance element 104b in place relative to the spine, as shown in FIG. 4J. Opposing compliance members 104a and 104b on opposite sides of spinous processes SP are oriented in opposite directions. Each compliance element 104a, 104b is coupled with their respective tether 102a, 102b and has a respective fastening mechanism or fastening element 106a, 106b. Fastening mechanism 106a, 106b are configured to couple with the tether 102a, 102b of the opposing compliance member 104a, 104b. For example as shown in FIG. 4K, tether 102a is advanced through compliance member 104b and is coupled with fastening mechanism 106b while tether 102b is advanced through compliance member 104a and is coupled with fastening mechanism 106a. Except for their orientation, compliance members 104a and 104b are identical. One of skill in the art will appreciate that the tether may enter and exit the fastening mechanism in a number of different directions and configurations, and FIG. 4K merely is one exemplary embodiment.

Fastening mechanism 106 may comprise a driver feature 108. As shown in FIG. 4L, the driver feature is adapted to receive a rotating driver tool RT. The driver feature may be a Phillips head, a slotted flat head, a Torx head, a hex head, or the like. Rotation of tool RT, which may be either clockwise or counter-clockwise, changes the configuration of fastening mechanism 106 so as to lock and secure tether 102 in place. This forms a continuous, multi-component tether structure or constraint 110 which couples two spinous processes SP together, as shown in FIG. 4M. Compliance elements 104a, 104b are used to control flexion between spinous processes SP while tethers 102a, 102b and respective fastening mechanisms 106a, 106b contribute to coupling the spinous processes SP together. Depending on the location of the perforations P1 and P2 and the lengths of the compliance elements 104a, 104b, constraint 110 may couple more than two spinous processes SP together. In general, compliance elements 104a, 104b comprise spring-like elements which will elastically elongate as tension is applied through tethers 102a, 102b in an axis generally parallel to the spine. As the spinous processes or spinous process and sacrum move apart during flexion of the constrained spinal segment, the superior tether 102a and inferior tether 102b will also move apart. Compliance elements 104a, 104b each include spring-like elements which will elastically resist the spreading with a force determined by the mechanical properties of the spring-like element. Thus, constraint 110 provides an elastic resistance to flexion of the spinal segment beyond the neutral position. Constraint 110 is often configured to provide a tensile resistance to spinal flexion, i.e., separation of the spinous processes, in the range from 7.5 N/mm to 25 N/mm, often from 10 N/mm to 15 N/mm The resistance to segmental extension may be below 3 N/mm or even below 0.5 N/mm Constraint 110 may also be adjustable in certain dimensions to allow tightening over the spinous processes or spinous process and sacrum when the spinal segment is in a neutral position. Other, related tether embodiments and joining methods are disclosed in U.S. patent application Ser. No. 12/106,103 (Attorney Docket No. 026398-000410US), U.S. Patent Publication No. 2008/0009866 (Attorney Docket No. 026398-000140US), U.S. Patent Publication No. 2008/0108993 (Attorney Docket No. 026398-000150US), U.S. patent application Ser. No. 12/106,049 (Attorney Docket No. 026398-000151US) and U.S. Provisional Patent Application No. 60/936,897 (Attorney Docket No. 026398-000400US), each of which, the entire contents are incorporated herein by reference.

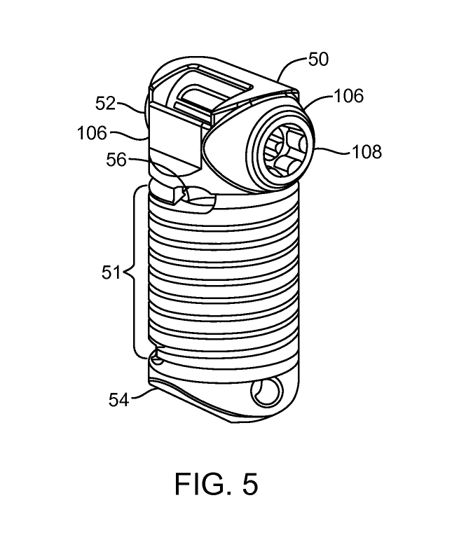

FIG. 5 illustrates an exemplary embodiment of a spring-like element 50 of compliance member 104a, 104b. Spring-like element 50 is generally similar to the spring-like elements disclosed in related, co-assigned U.S. patent application Ser. No. 12/106,103, the entire contents of which are incorporated herein by reference. Fastening mechanism 106 having a driver feature 108 is housed within spring-like element 50. Element 50 comprises a housing having a helical groove machined in the housing body to form the spring-like element. Element 50 includes an adjustable tether connector 52 and a fixed tether connector 54, both of which are preferably formed integrally or monolithically with the helical spring structure 51. Typically, the helical spring structure 51 and coupling portions of both tether connectors 52 and 54 will be formed from one piece of material, usually being a metal such as titanium, but optionally being a polymer, ceramic, reinforced glass or other composite, or other material having desired elastic and mechanical properties and capable of being formed into the desired geometry. In a preferred embodiment, spring-like element 50 is machined or laser cut from a titanium rod. Alternatively, a suitable polymeric material will be polyetherether ketone (PEEK). Other features may be built into the spring-like element 50, such as a stress relief hole 56. Components that compose the adjustable tether connector may potentially include a roller and a lock-nut; such components could be made from the same material as the element 50 and adjustable tether connector (e.g. titanium components if the spring-like element 50 is titanium), or they could be made from a different material (e.g. injection molded PEEK). The exterior of the spring-like element 50 may be covered with a protective cover, such as a sheath fabricated from an elastomer, polymer or other suitable material. The sheath may be placed over the body of the spring-like element 50 in order to prevent the intrusion of tissue and body fluids into the spaces between the turns of the coil and interior of the element.

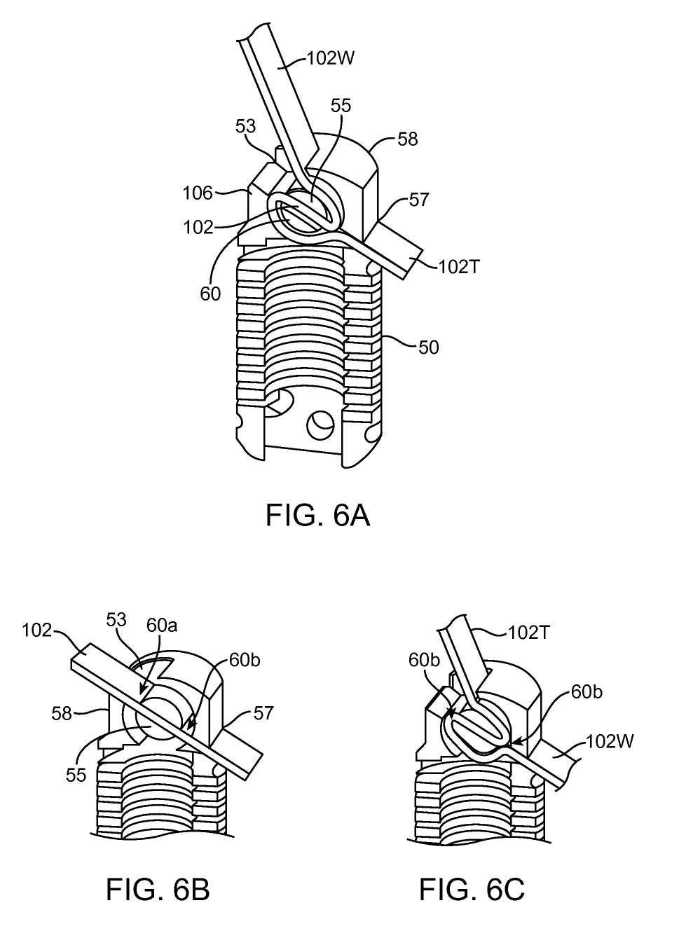

FIG. 6A shows a cross-section of spring-like element 50 having tether 102 locked therein. Tether 102 enters and exits the housing 58 of fastening mechanism 106 through entry aperture 53, then it passes through central channel 55, winds around roller 60 and the inside surface of housing 58, and finally exits through exit aperture 57. Roller 60 is housed within central channel 55 and is rotatable within tension element 50. Roller 60 is often substantially cylindrically shaped but may also have other shapes, for example, an eccentric shape. A round symmetrical roller will allow the tether 102 to spool evenly from both the working end and the tail end of the tether 102, while an eccentrically shaped roller will result in uneven spooling. The housing 58 of fastening mechanism 106 may be formed integrally with spring-like element 50 or may be separate.

During a procedure similar to the one described with reference to FIGS. 4A-4M, tether 102 is advanced through top aperture 53, central channel 55 and roller 60, and out through bottom aperture 57. As shown in FIG. 6B, top aperture 53, central channel 55, and bottom aperture 57 are aligned so permit easy passage of tether 102 therethrough. Roller 60 includes two side apertures 60a, 60b. Prior to the locking of the tether, entry aperture 53, side apertures 60a and 60b and exit aperture 57 are all aligned along a common axis. To provide such alignment, roller 60 may include an alignment feature such as a pin or shoulder. Thus, the roller 60 may be rotated until stopped by the pin or shoulder, thereby ensuring alignment of all the apertures. Once tether 102 is advanced through, roller 60 is rotated, via driver feature 108, thus creating a friction-based interference fit between roller 60, the inside surface of the housing and the tether 102. As shown in FIG. 6C, the fastening mechanism is rotated approximately 180° to create this fit. The rotation of the roller creates a tortuous path for the tether as it passes between side apertures 60a, 60b. The rotation may retract the working end 102w and tail end 102t of tether 102, sometimes of different lengths, inward toward roller 60. Offsetting roller 60 from its axis of rotation by using an eccentrically shaped roller changes the amount of tether drawn from either side. The roller may also be rotated a selected amount in order to draw a desired amount of the tether into the roller. For example, the roller may be rotated from about V4 turn to two or more complete revolutions. Thus, not only will the locking mechanism secure the tether in position, but it may also be used to help adjust length or tension of the tether.

A friction-based interference fit is advantageous because the range along the tether to which the mechanism can attach is continuous, rather than in discrete increments of non-friction mechanisms such as teeth, hooks, loops, and the like. Thus, forces between roller 60 and tether 102 are distributed along a longer portion of tether 102. Additionally, high clamping forces are not required. Thus, the risk that any specific point of contact will abrade, wear, or will otherwise be damaged is minimized. Furthermore, in contrast with other mechanisms that require high clamping forces, the discrete rotation of a tool is easier and more repeatable to perform during surgery.

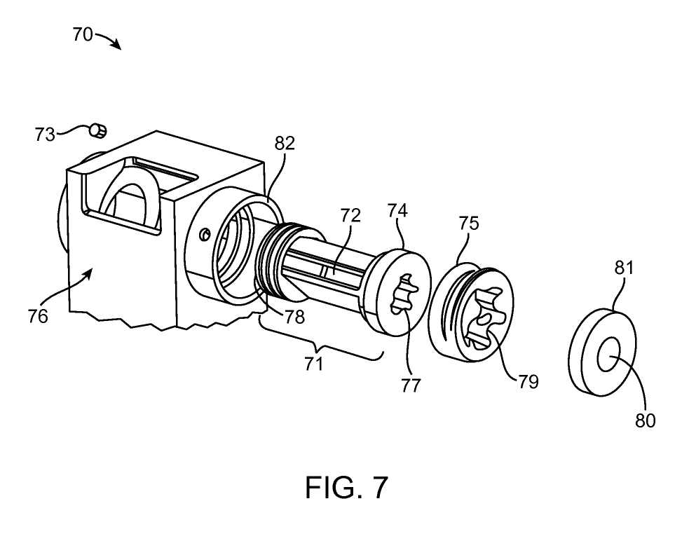

After the tether is secured, roller 60 is then locked in place. Various means may be provided to lock roller 60 in place within housing 58. Roller 60 and/or the inner surface of housing 108 may include male or female threads which engage the two elements together. The threads may be partially deformed, thereby helping to secure the roller element with the housing. Alternatively, a pin 73 may be coupled to housing 58 and roller 60 may comprise a groove adapted to receive pin 73. Another possibility is that housing 58 may include a flange adapted to retain roller 60. A set screw as described below with reference to FIG. 7 may also be provided to lock roller 60 in place. Rotation of roller 60 in the opposite direction unwinds tether 102 from roller 60 and reduces the interference fit. Roller 60 and/or housing 58 may further include a position indicator, such as detents or calibration marks, to provide visual, tactile, or audible feedback to an operator on the relative position of the roller with respect to housing 58.

FIG. 7 shows an exploded view of an exemplary fastening mechanism 70 that uses a locking set screw 75 to lock roller 76 in place. Roller 71 is generally similar to roller 60. It is positioned within housing 76 and includes slots 72 for a tether to be advanced through. Roller 71 has threads 78 on one end that may be threadably engaged with the housing 76. Roller 71 also has a shoulder 74 and includes driver features 77. Shoulder 74 is adapted to be engagable with locking set screw 75 and housing 76. After roller 71 has been rotated to lock and secure a tether in place, set screw 75 is set in a position to engage roller 71 with housing 76 and hold it in position relative to housing 76. Shoulder 74, set screw 75, and/or housing 76 have threads to allow such engagement. The threads may be partially deformed, thereby further securing the locking member with the housing. The threads prevent the roller 71 from unrolling thereby allowing release of the tether. Set screw 75 may comprise driver features 79 to allow rotation of the set screw. Driver features 77 of roller 71 and driver features 79 of set screw 75 each are adapted to receive a tool so as to permit rotation thereof. The driver features 77, 79 may be a Phillips head, a slotted flat head, a Torx head, a hex head, or the like. Driver features 79 of set screw 75 may comprise an aperture large enough to permit access to roller 71 with a tool permit rotation of roller 71 with a tool while set screw 75 is engaged with housing 76. An optional end cap 81 having a central aperture 80 may be positioned adjacent the set screw 75 and welded, bonded or otherwise affixed to the outer rim 82 of the housing 76 so as to capture all the components forming an inseparable assembly. The aperture 80 is sized to allow access to rotation of the set screw. This is desirable since it prevents parts from falling out during use and also provides a device which is easier to use since assembly is not required. In preferred embodiments, the assembly may not be disassembled without breaking or otherwise damaging the device. In other embodiments, the assembly may be disassembled without damaging the device.

One advantage of the roller locking mechanisms disclosed herein is that the tether is not deformed in planes in which it lies. The tether may be folded or rolled in a plane transverse to the planes in which it lies. This is desirable since it minimizes the possibility of twisting or tangling of the tether and also reduces wear and tear.

While the exemplary embodiments described above illustrate a fastening mechanism that is coupled with a spring-like compliance member, one will appreciate that the fastening 25 mechanism may be used independently of a spring or other internal fixator. Other uses may include applications where a tether is secured with a knot, crimped or the like.

The flexion limiting device described above is a promising treatment for lower back pain or instability. Additionally, it may be used with other treatments such as spinal fusion that can further provide a good clinical outcome for patients suffering from back pain. Preferred embodiments of the tether structure will be sized to fit in the treatment space and also will have a porosity that allows body fluids such as blood to flow in and out of the region of spinal fusion. The tether structure may be used alone, or in combination with more traditional spinal instrumentation that often accompanies spinal fusion procedures. Spinal instrumentation may include pedicle screws that are polyaxial or monoaxial, and the spinal rods may be dynamic rods or static rods. In other embodiments, a tether alone may be used to constrain flexion of the spine and to facilitate spinal fusion. The below describes exemplary usage of a tether based flexion limiting device in conjunction with spinal fusion.

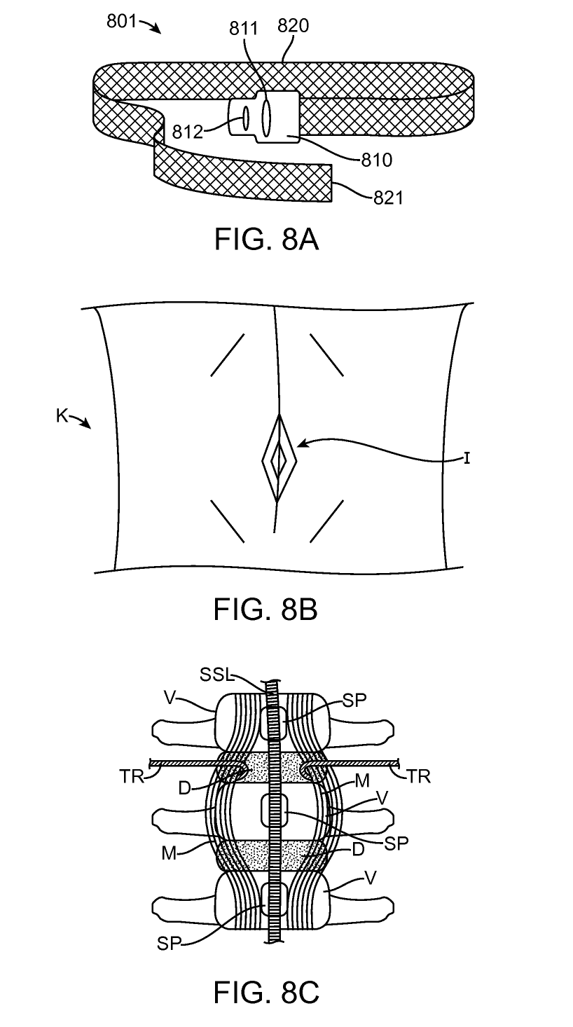

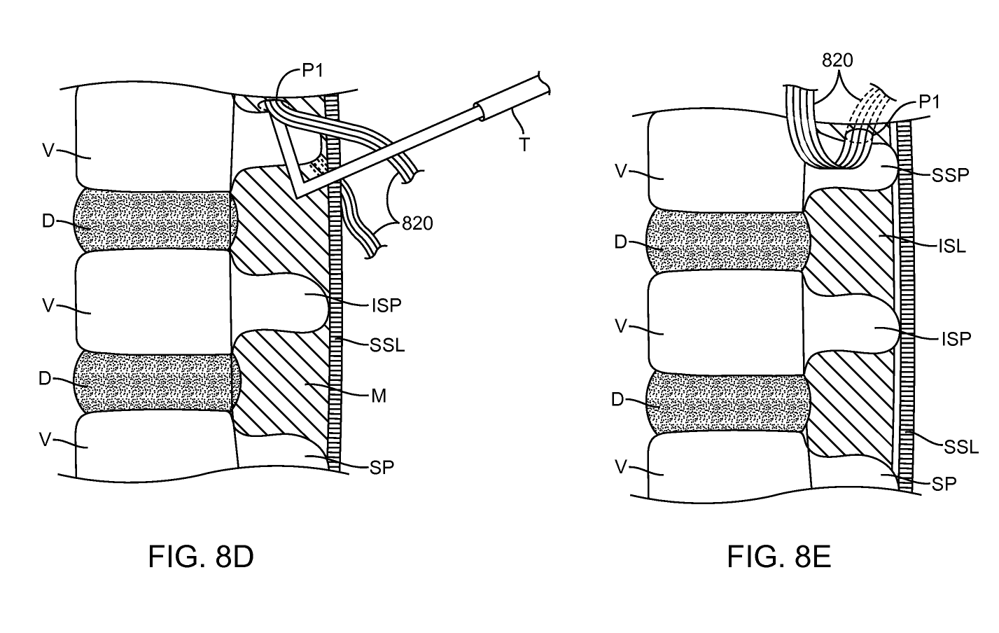

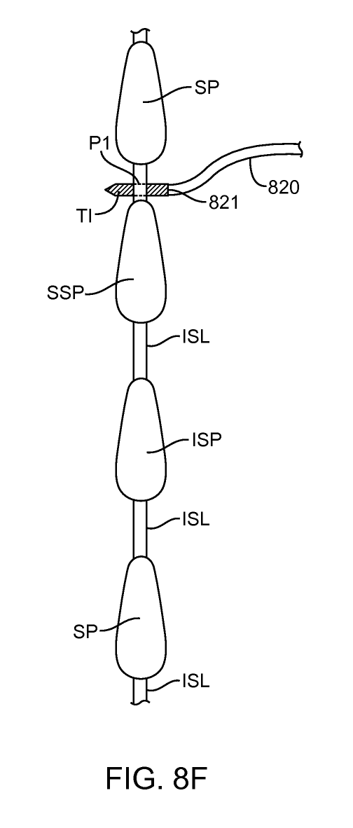

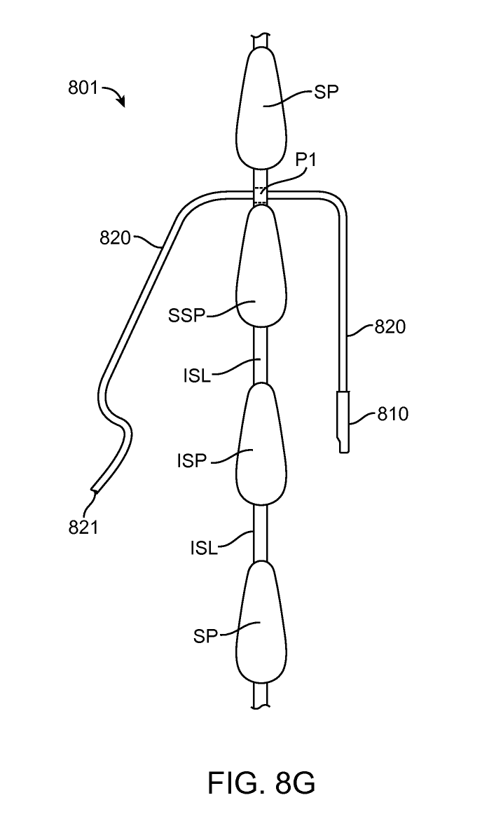

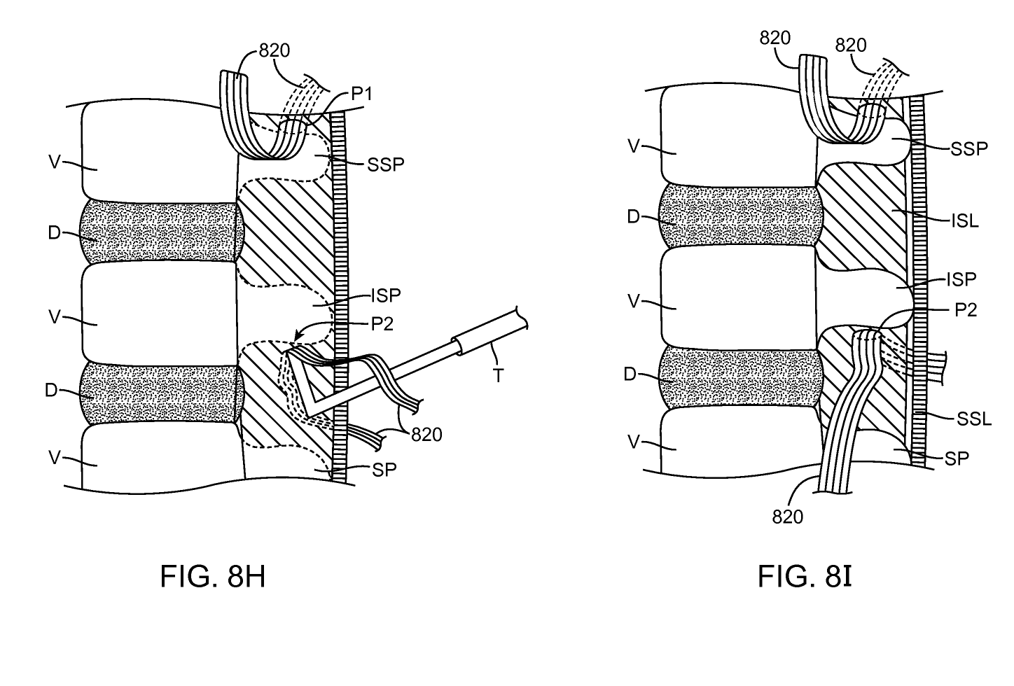

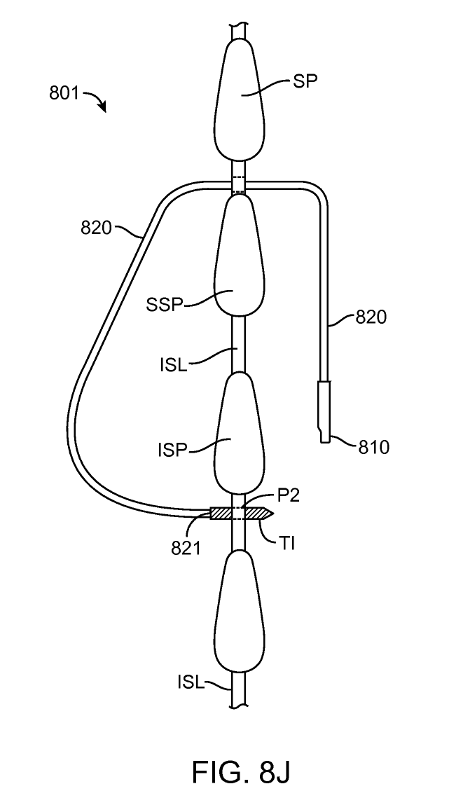

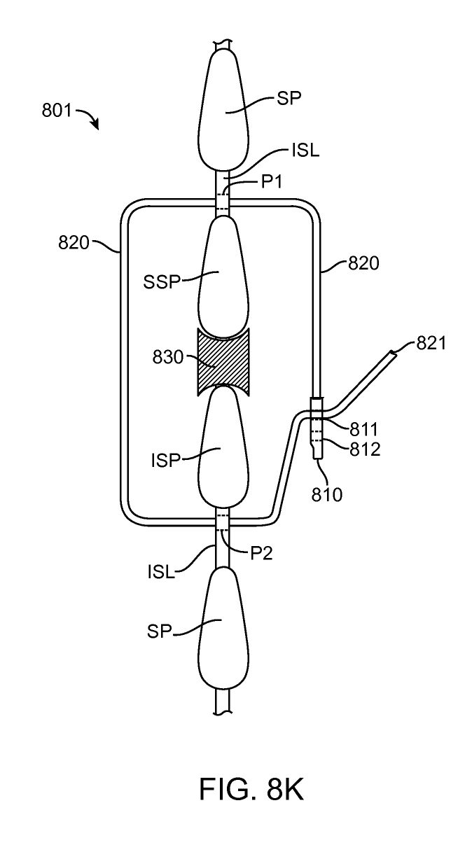

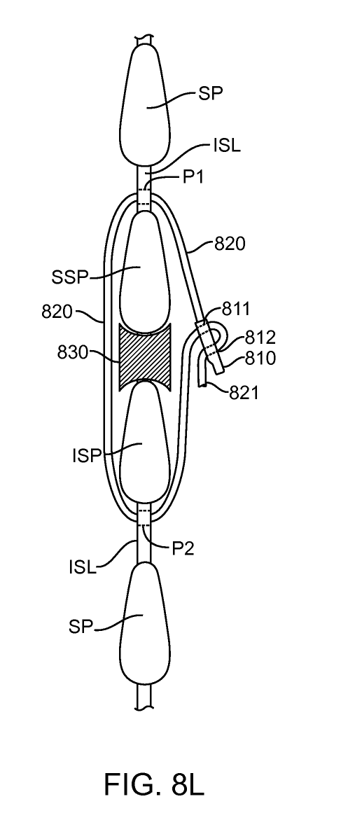

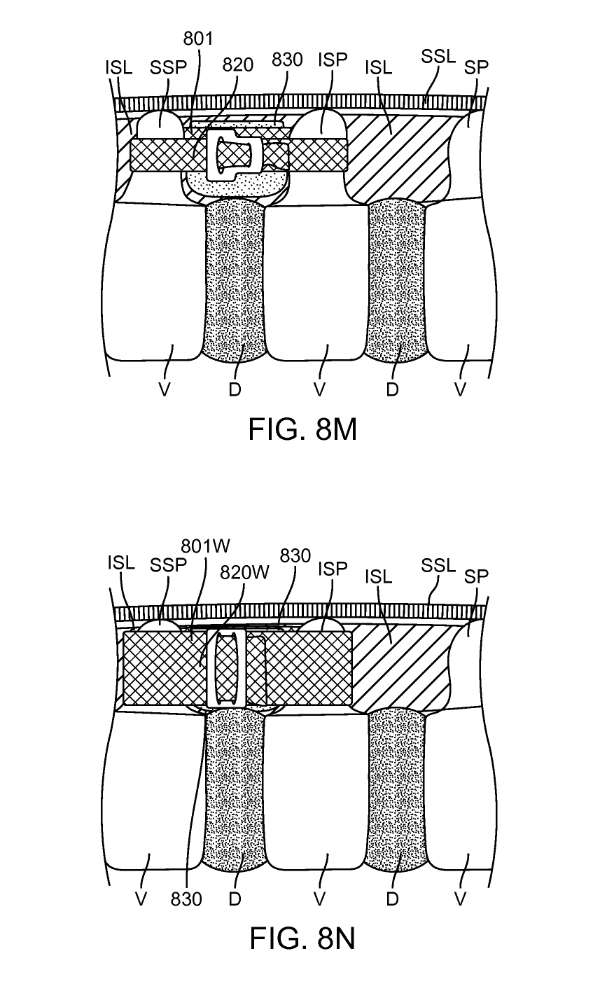

FIG. 8A shows a spinal implant or spinal process constraint 801 which can be used to constrain flexion of the spine and to facilitate spinal fusion. The spinal process constraint 801 is a structure similar to a belt and comprises a connector or buckle 810 coupled to a flexible tether structure 820 having a free end 821. The flexible tether structure 820 will typically be porous, e.g, comprise a flexible porous textile. The buckle 810 comprises a first slot 811 and a second slot 812. The connector or buckle 810 may also instead comprise any of the tether locking and fastening mechanisms described above in reference to FIGS. 5-7. As shown in FIG. 8A, the spinal process constraint 801 is in an open configuration. The free end 821 of the flexible tether structure 820 can be tightened and secured to connector 810. In the example of FIG. 8A, the free end 821 can be passed through the first slot 811 and the second slot 812 to close the spinal process constraint 801 into a closed configuration and also to tighten the entire structure.

FIGS. 8B-8N illustrate an exemplary surgical method of implanting the spinal process constraint 801. This exemplary surgical method is similar in many respects to the surgical method described above in reference to FIGS. 4A-4M. One of the first steps to surgically implant a spinal implant is to make an incision to access the spinal area of interest. FIG. 8B shows the lumbar region of back K after an incision I has been made through the patient's skin.

FIG. 8C shows multifidus muscle and tendon M having been retracted with retraction tools TR to expose the spinous processes. After the incision has been made, surgical procedures such as a neural decompression may optionally be performed.

After the incision has been made and any other procedures such as a neural decompression have been performed, a piercing tool T having a sharp distal end may be used to access and pierce the interspinous ligament ISL while avoiding the supra spinous ligament SSL, creating an interspinous ligament perforation P1 superior of the first spinous process SSP of interest. This surgical approach is desirable since it keeps the supra spinous ligament intact and minimizes damage to the multifidus muscle and tendons and other collateral ligaments. As shown in FIG. 8D, from the right side of the spine, tool T accesses and pierces the interspinous ligament ISL adjacent of the first spinous process SSP of interest. The distal end of tool T is shown in dotted line. Alternatively, tool T may access and pierce the interspinous ligament ISL from the left side instead. The distal end of tool T is coupled with tether structure 820, parts of which are also shown in dotted line. In addition to accessing and piercing the interspinous ligament ISL, piercing tool T also advances or threads tether structure 820 through perforation P1. As shown in FIG. 8E, tool T is then removed, leaving tether structure 820 positioned through perforation P1. Multifidus tendon and muscle M is not shown in FIGS. 8D and 8E so that other elements are shown more clearly.

FIG. 8F is a posterior view of a section of the spine after the above steps have been performed. Often times, the distal tip TI of tool T is detachable. As shown in FIG. 8F, after tool T accesses and pierces the interspinous ligament ISL with distal tip TI, distal tip TI is detached from tool T and is left in place in perforation P1 (shown in dotted line) above the first spinous process SSP of interest. Tether structure 820 lags behind tip TI. In some cases, distal tip TI may fully pierce through interspinous ligament ISL. In these cases, distal tip TI has passed through the interspinous ligament ISL while a portion of tether 820 is left in place in perforation P1.

After tip TI or a portion of tether structure 820 is left in place in perforation P1, another tool may couple with tip TI and pull tip TI such that it drags tether structure 820 to its appropriate position relative to the spine, as shown in FIG. 8G.

The steps of accessing the ISL, piercing the ISL, and threading tether structure 820 through a perforation are then repeated for the opposite, lateral side of the spine for an adjacent spinous process ISP, inferior of the first superior spinal process SSP of interest. As shown in FIGS. 8H and 8I, tool T accesses the interspinous ligament ISL from the left side of the spinal midline and pierces the interspinous ligament ISL, creating a second perforation P2 located inferior of a second spinous process of interest, labeled as inferior spinous process ISP. As shown in FIG. 8H, the inferior spinous process ISP of interest is directly adjacent to and inferior of the first superior spinous process SSP of interest. However, it is entirely possible to perform the described procedure starting with the inferior spinous process ISP first instead of the superior spinous process SSP, for example, perforation P2 may be created before perforation P1. It is also possible that there may be a gap of one or more spinous processes SP between the spinous processes of interest. Multifidus tendon and muscle M is not shown in FIGS. 8H and 8I for clarity of the other shown elements.

As shown in FIGS. 8H, 8I, and 8J, like with the steps shown in conjunction with the first piercing, tether structure 820 is pierced through perforation P2 and left in place along with distal tip TI of the tool T. Another tool, such as a pair of forceps, is then used to grasp distal tip TI to pull the tether structure 820 through the perforation P2. As shown in FIG. 8K, a portion of the interspinous ligament ISL as well as portions of the spinous processes and lamina can be removed and bone graft 830 placed in the space created. A fusion cage may be additionally placed between the spinous processes for structural support and bone graft containment. As shown in FIG. 8L, the distal end 821 of the tether structure 820 may be fed through the first slot 811 and then the second slot 812 of the buckle 810 to tighten the spinous process constraint 801 over the superior spinous process SSP, the bone graft 830, and the inferior spinous process ISP. A tool, such as a pair of forceps or clamps, may be used for this step.

FIG. 8M shows a side view of the spinous process constraint 801 tightened over the superior spinous process SSP, the bone graft 830, and the inferior spinous process ISP. To facilitate turning the bone graft 830 into a solid fusion mass, the bone graft 830 should be loaded and exposed to blood. Thus, the tether structure 820 of the spinous process constraint 801 will typically be porous and wide enough to contain and hold the bone graft 830 in place. The width of the strap may range from 5 mm to 25 mm In some embodiments, for example as shown in FIG. 8N, the tether structure 820W will be wide enough such that it substantially covers the majority of the surface of the spinous processes which it wraps around, thereby improving the ability of the tether structure 820W to restrict flexion and also to hold the bone graft 830 in place. The tether structures 820, 820W may be made of a porous textile fiber, e.g., a textile with an open weave or braid construction. The tether structure is tensioned to provide a compressive force on the bone graft via the spinous processes to facilitate development of a solid fusion mass.

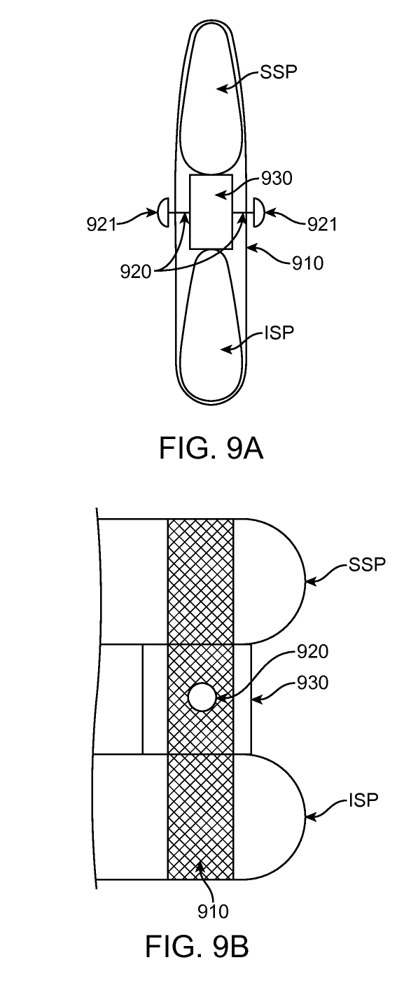

Additional structures that help a spinal implant hold an encircled bone graft or fusion cage in place may also be provided. These structures may be used or implanted using methods similar to that described above with reference to FIGS. 8A-8N. FIGS. 9A and 9B show a posterior view and a side view, respectively, of a superior spinous process SSP, a fusion cage or bone graft 930, and an inferior spinous process ISP encircled by a strap cerclage or tether structure 910. After the tether structure 910 has been tightened over the desired anatomy, one or more fasteners such as spikes 920 on either side of the bone graft 930 are pierced through the tether structure 910 and the bone graft 930. The one or more fasteners 920 may comprise a head or cap to retain the fastener. The fastener 920 may be a single component that pierces both sides of the tether structure and bone graft, or separate components on the right and left sides.

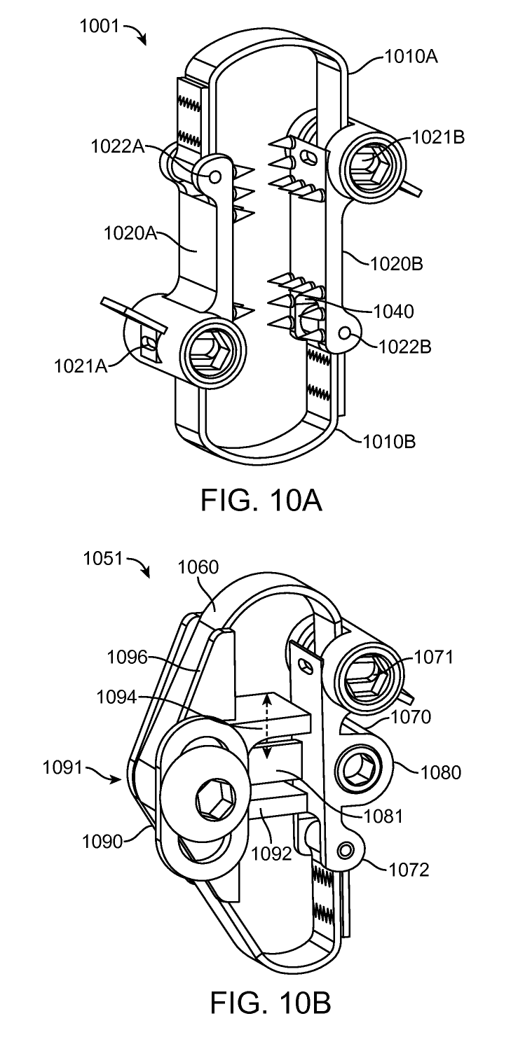

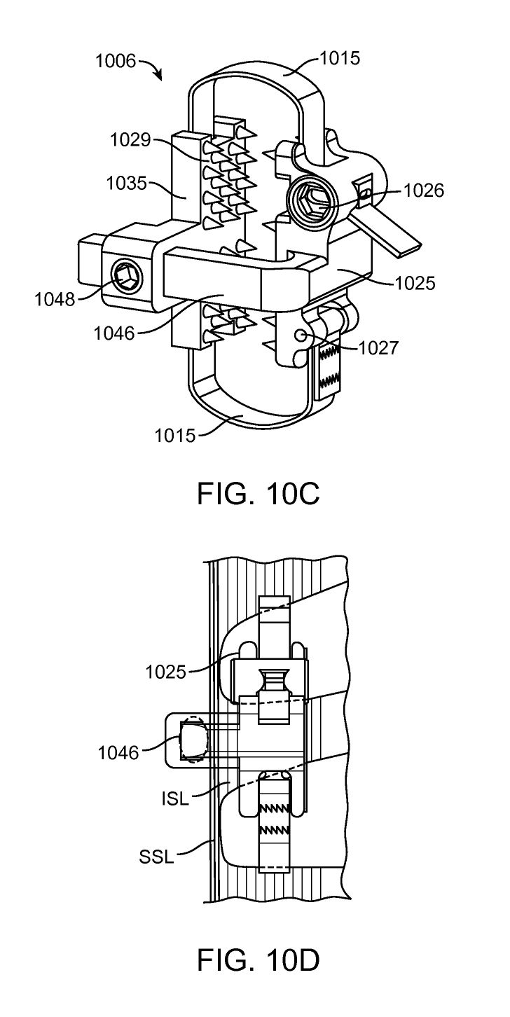

The spinal implant or spinal process constraint 801 shown in FIGS. 8A-8N comprises a single strap and a single buckle or connector. The present invention also provides spinal implants or spinous process constraints with alternative structures. FIG. 10A shows a spinal implant or spinal process constraint 1001 for restricting flexion. The spinal implant or spinous process constraint 1001 comprises two straps or tethers, a first tether 1010A for placement over a superior spinous process and a second tether 1010B for placement over an inferior spinous process. The spinous process constraint 1001 further comprises a first plate 1020A and a second plate 1020B, each of which comprise spikes 1040 disposed on the inward-facing sides of the first plate 1020A and the second plate 1020B to facilitate their purchase on the bone of the spinous processes. (Spikes may also be provided for the buckle 810 of the spinal processes constraint 801 to facilitate its purchase on the bone of the spinous processes, with the spikes disposed on the inward-facing sides of the buckle 810.) The first tether 1010A is coupled to the first plate 1020A through a fixed strap connection 1022A. The second tether 1010B is coupled to the second plate 1020B through a fixed strap connection 1022B. Each of the plates 1020A, 1020B also comprise strap locking mechanisms 1021A and 1021B, respectively, through which the tethers 1010A and 1010B can be passed through and locked. Strap locking mechanisms 1021A and 1021B may comprise any of the tether locking and fastening mechanisms described above in reference to FIGS. 5-7. The spinous process constraint structure of FIG. 10A-B functions as that of FIGS. 8-9: the tether structure applies a compressive force to bone graft via the spinous processes, facilitating development of a solid fusion mass. The plate features provide additional grip on the spinous process, and contain the bone graft between the spinous processes. A transverse fastener like element 920 in FIGS. 9A-B may likewise be utilized to secure the bone graft to the plates.

FIG. 10B shows a spinal implant or spinal process constraint 1051 for limiting flexion. The spinal implant or spinal process constraint 1051 comprises a single strap or tether structure 1060 for encircling both a superior and an inferior spinous process. The spinal process constraint 1051 further comprises a first plate 1070 comprising a fixed strap attachment 1072 through which the single strap or tether structure 1060 is attached, a strap locking mechanism 1071 through which the single strap or tether structure 1060 can be passed through and locked, and a cross-member locking element 1080. The strap locking mechanism 1071 may comprise any of the tether locking and fastening mechanisms described above in reference to FIGS. 5-7. The spinal process constraint 1051 further comprises a second plate 1091 over which the single strap or tether structure 1060 is wrapped around. The second plate 1091 further comprises an adjustable cross member 1096 and a fixed cross member 1092. The adjustable cross member 1096 and the fixed cross member 1092 traverse the gap between the first plate 1060 and the second plate 1091. The adjustable cross member 1096 and the fixed cross member 1092 also limit extension of the two spinal processes which the constraint 1051 is wrapped around and provide for containment of bone graft material between the spinous processes. The second plate 1091 further comprises an adjustable cross-member locking element 1091, which is shown as a screw head clamping onto a flange with an ob-round slot, which can be used to adjust the distance between the fixed cross member 1092 and the adjustable cross member 1092. Such distance adjustment can also be applied to maintain a desired distance between the two spinous processes which the constraint 1051 is wrapped around or be used to accommodate different interspinous spacings. The spinous process constraint 1051 further comprises a central cross member 1081 connecting the first plate 1070 and the second plate 1091 together. The first plate 1070 further comprises a cross-member locking element 1080 which can be used to adjust the width of the spinal process constraint 1051 by adjusting how much the central cross member 1081 extends. The central cross member 1081 is typically disposed ventral of the adjustable cross-member 1096 and the fixed cross member 1092 so as to define a concavity into which a bone graft may be placed.

FIG. 10C shows a similar, spinal implant or spinous process constraint with tethers circumscribing the spinous processes, applying a compressive force to bone graft via the spinous processes, and means for bone graft containment. The spinous process constraint 1006 of FIGS. 10C-10D have a cross-piece 1046 that straddles the spinal midline dorsal to the super spinous ligament SSL/interspinous ligament ISL complex as shown in the side view of FIG. 10D. The spinous process constraint 1006 comprises a first plate 1025, a second plate 1035, and a cross-piece 1046 connecting the first plate 1025 with the second plate 1035. The cross-piece 1046 sits dorsal to the first plate 1025 and the second plate 1035 such that the cross-piece 1046 can straddle the spinal mid-line dorsal to the super spinous ligament SSL/interspinous ligament ISL complex. By using this positioning, resection of the interspinous ligament, super spinous ligament, and spinous processes can be avoided if desired, or bone ingrowth during interspinous or interlaminar fusion can be accommodated. The first plate 1025 of the spinal process constraint 1006 further comprises a fixed connector 1027 through which a single strap or tether connector 1015 is fixedly attached to the first plate 1025 and a strap locking mechanism 1026 through which the single strap or tether connector 1015 can be passed through and locked in place. The strap locking mechanism may comprise any of the tether locking and fastening mechanisms described above in reference to FIGS. 5-7. The first plate 1025 and the second plate 1035 also each comprise spikes 1029 disposed on the inwardly-facing sides of the first plate 1025 and the second plate 1035 to facilitate the purchase of the first plate 1025 and the second plate 1035 onto the spinous processes. The cross-piece 1046 is fixedly attached to the first plate 1025 and moveably coupled to the second plate 1035. A cross-piece locking element or set screw 1048 fixedly coupled to the second plate 1035 can be used to lock the cross-piece 1046 in place. Locked in place, the cross-piece 1046 can provide an additional clamping force to the first plate 1025 and the second plate 1035 to hold the spinal process constraint 1006 in place relative to the spinal processes.

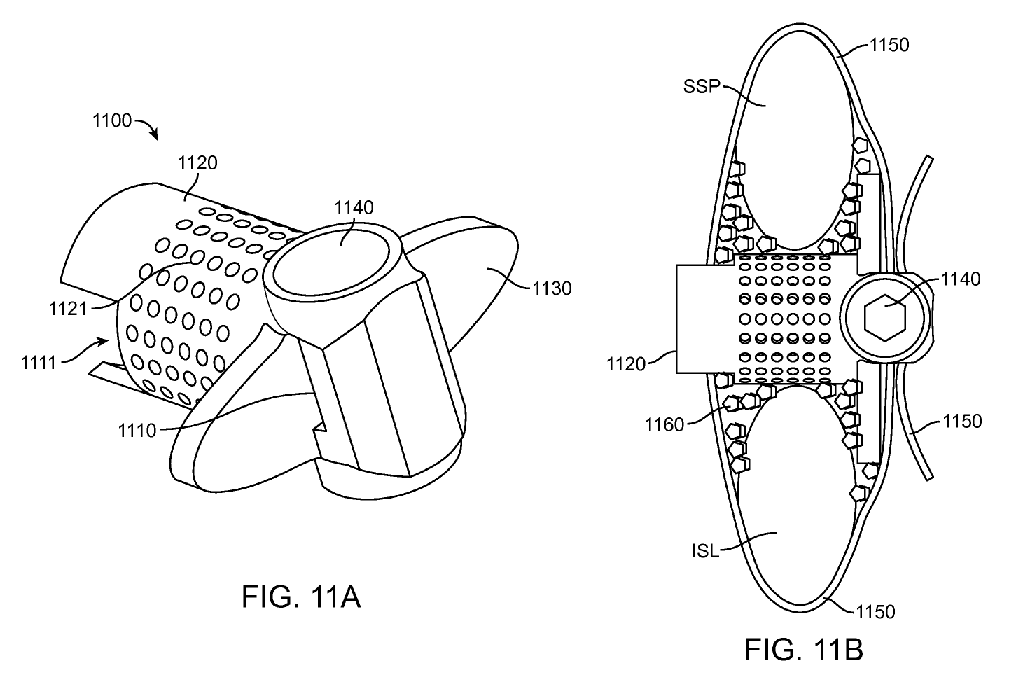

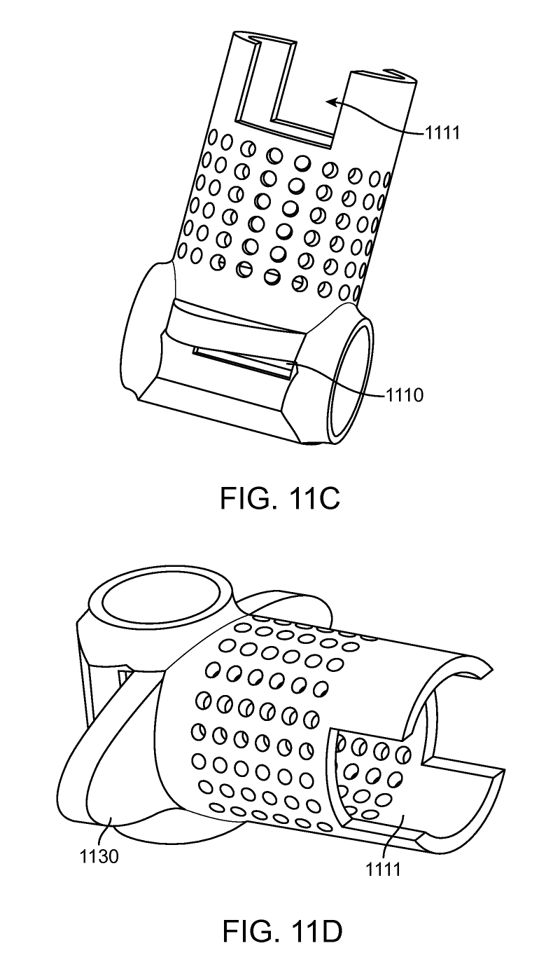

The present invention also provides spinous process cages for providing structural support while being able to contain bone graft in order to potentially promote bony fusion between adjacent spinous processes or other structures. FIG. 11A shows an exemplary spinous process cage 1100 having structures, i.e. strap containment elements 1110 and 1111, for accommodating a strap or tether structure, for example those described above in reference to FIGS. 8A-10D. The spinous process cage 1100 can be used in the method described above with reference to FIGS. 8A-8N or similar methods. The spinous process cage 1100 further comprises a main cylindrical body 1120 having fusion pores 1121, an ipsilateral medial-lateral stop and tension flexure 1130, and a tightening feature 1040, e.g., a set screw or fastening mechanism described above in reference to FIGS. 5-7, for locking a strap or tether structure in place. FIG. 11B shows the spinous process cage 1100 facilitating the wrapping of a strap or tether structure 1150 around a superior spinous process SSP, an inferior spinous process ISP, and a plurality of bone graft material 1160 therebetween. FIGS. 11C and 11D show the spinous process cage 1100 from different angles. As shown in FIGS. 11C and 11D, strap containment element 1110 is a cut out from main cylindrical body 1120. The cut-out is of a size and shape to receive and contain the strap or tether structure 1150 which contains the bone graft material 1160 within the spinous process cage 1100. As shown in FIGS. 11C and 11D, the ipsilateral medial-lateral stop and tension flexure 1130 comprise wing structures which are typically flexible and used to catch up any potential slack in the strap or tether structure 1160.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A system for fusing a spine, said system comprising:
a flexion limiting tether having a superior portion, and an inferior portion, wherein the superior portion of the tether is coupled to a superior portion of the spine, and the inferior portion of the tether is coupled to an inferior portion of the spine thereby constraining flexion of the spine; and
bone graft for fusing the superior and inferior portions of the spine together, the bone graft disposed between the superior and inferior portions of the spine, wherein the tether has a width suitable for holding the bone graft in a mass disposed between the superior and inferior portions of the spine.

2. The system of claim 1, wherein the tether has a porosity suitable to allow body fluids to pass therethrough so that material of the bone graft forms a solid mass.

3. The system of claim 1, wherein the superior portion of the spine comprises a superior spinous process and the inferior portion of the spine comprises an inferior spinous process.

4. The system of claim 3, wherein the flexion limiting tether is wide enough to cover a majority of the lateral surfaces of the superior and inferior spinous processes.

5. The system of claim 1, wherein the system further comprises a connector, and wherein the flexion limiting tether consists essentially of a single strap having a free end and a fixed end, the fixed end being fixedly coupled to the connector and the free end being adjustably coupled to the connector such that the tether can be tightened over the superior and inferior portions of the spine.

6. The system of claim 5, wherein the connector comprises an inward facing surface having one or more spikes adapted to facilitate purchase of the connector onto the bone of the superior or inferior portion of the spine.

7. The system of claim 5, wherein the system further comprises a plate adapted to be disposed on the other side of a spinal midline from the connector.

8. The system of claim 7, wherein the flexion limiting tether is wrapped over the plate.

9. The system of claim 7, wherein the plate comprises an inward facing surface having one or more spikes adapted to facilitate purchase of the plate onto the bone of the superior or inferior portion of the spine.

10. The system of claim 7, wherein the plate comprises one or more cross-members adapted to traverse the spinal midline to couple to the connector.

11. The system of claim 10, wherein the one or more cross-members are dorsal of the connector and the plate.

12. The system of claim 10, wherein the one or more cross-members comprise a central cross-member for adjusting the distance between the connector and the plate.

13. The system of claim 10, wherein the one or more cross-members comprise a fixed cross-member and an adjustable cross-member, the position of the adjustable cross-member relative to the fixed cross-member being adjustable so as to adjust the distance between the adjustable cross-member and the fixed cross-member.

14. The system of claim 13, wherein the distance between the adjustable cross-member and the fixed cross-member can be adjusted to distract the superior and inferior portions of the spine.

15. The system of claim 1, wherein the superior portion of the tether comprises a first strap and the inferior portion of the tether comprises a second strap distinct from the first strap.

16. The system of claim 15, wherein the first strap comprises a fixed end and a free end, and the second strap comprises a fixed end and a free end, and
wherein the system further comprises a first connector and a second connector, the fixed end of the first strap being fixedly coupled to the first connector and the free end of the first strap being adjustably coupled to the first connector such that the first strap can be tightened over the superior portion of the spine, the fixed end of the second strap being fixedly coupled to the second connector and the free end of the second strap being adjustably coupled to the second connector such that the second strap can be tightened over the inferior portion of the spine.

17. The system of claim 16, wherein the first connector and the second connector are disposed on opposite sides of a spinal midline.

18. The system of claim 16, wherein the first connector comprises an inward facing surface having one or more spikes adapted to facilitate purchase of the first connector to bone of the superior portion of the spine.

19. The system of claim 16, wherein the first connector comprises an inward facing surface having one or more spikes adapted to facilitate purchase of the first connector to bone of the superior portion of the spine.

20. The system of claim 16, further comprising one or more cross-members adapted to traverse the spinal midline to couple the first connector to the second connector.

21. The system of claim 20, wherein the one or more cross-members are dorsal of the first connector and the second connector.

22. The system of claim 20, wherein the one or more cross-members comprise a central cross-member for adjusting the distance between the first connector and the second connector.

23. The system of claim 20, wherein the one or more cross-members comprise a fixed cross-member and an adjustable cross-member, the position of the adjustable cross-member relative to the fixed cross-member being adjustable so as to adjust the distance between the adjustable cross-member and the fixed cross-member.

24. The system of claim 23, wherein the distance between the adjustable cross-member and the fixed cross-member can be adjusted to distract the superior and inferior portions of the spine.

25. The system of claim 1, further comprising a fusion cage for holding the bone graft in place.

26. The system of claim 25, wherein fusion cage comprises a cylindrical main body having a plurality of pores to allow body fluids to pass therethrough so that the material of the bone graft can form a solid mass.

27. The system of claim 1, further comprising one or more fasteners for piercing through the flexion limiting tether and into the bone graft to hold the flexion limiting tether in place relative to the bone graft.

28. A method for fusing a spine, said method comprising:
providing a tether;
coupling the tether to a superior portion of the spine and an inferior portion of the spine, wherein the tether constrains flexion of the spine;
providing bone graft and disposing the bone graft between the superior and inferior portions of the spine; and
constraining the bone graft with the tether so that the bone graft is held in a mass; and
tightening the tether to apply a compressive force to the bone graft via the superior and inferior portions of the spine.

29. The method of claim 28, further comprising allowing body fluids to pass through the tether into contact with the bone graft thereby allowing the bone graft to form a solid mass.

30. The method of claim 28, wherein the superior portion of the spine comprises a superior spinous process and the inferior portion of the spine comprises an inferior spinous process.

31. The method of claim 30, further comprising removing at least a portion of the interspinous ligament between the superior spinous process and the inferior spinous process prior to disposing the bone graft therebetween.

32. The method of claim 28, further comprising providing a connector for the tether, the connector being fixedly coupled to a fixed end of the tether and being adjustably coupled to an adjustable end of the tether, and adjusting the position of the adjustable end of the tether relative to the connector so as to loosen or tighten the tether over the bone graft and superior and inferior portions of the spine.

33. The method of claim 32, further comprising providing one or more spikes on an inward facing surface of the connector, the one or more spikes facilitating purchase of the connector to bone of the superior or inferior portion of the spine.

34. The method of claim 28, further comprising providing a fusion cage for holding the bone graft in place relative to the superior and inferior portions of the spine.

35. The method of claim 28, further comprising piercing the tether and bone graft with a fastener and leaving the fastener in place through the tether and bone graft to hold the bone graft in place relative to the tether.
